(19) United States
(12) Patent Application Publication
(10) Pub. No.: US 2026/0092302 A1
(43) Pub. Date: Apr. 2, 2026

(54) SYSTEMS AND METHODS FOR BIOMARKER DETECTION

(71) Applicant: Taudia, Inc., Palo Alto, CA (US)

(72) Inventors: Hien NGUYEN, San Jose, CA (US); Robert LIN, Palo Alto, CA (US)

(21) Appl. No.: 19/385,783

(22) Filed: Nov. 11, 2025

Related U.S. Application Data
(63) Continuation of application No. PCT/US25/48840, filed on Sep. 30, 2025.
(60) Provisional application No. 63/701,846, filed on Oct. 1, 2024.
(60) Provisional application No. 63/764,444, filed on Feb. 27, 2025.
(60) Provisional application No. 63/793,208, filed on Apr. 23, 2025.

(51) Int. Cl. C12Q 1/6804; C12Q 1/682; G01N 33/58

(52) U.S. Cl. CPC C12Q 1/6804; C12Q 1/682; G01N 33/582

(57) ABSTRACT

Provided herein are systems, compositions, and methods for detecting analytes (e.g., proteins, nucleic acid molecules, biomolecules, peptides, antibodies, biomarkers). In an aspect, a method for analyte detection, comprising: (a) providing: (i) a first probe coupled to a surface of a substrate, and (ii) said analyte; (b) binding said first probe coupled to said surface of said substrate and a second probe to said analyte, to generate a complex; (c) using said first probe and said second probe of said complex to generate a reaction product; and (d) detecting said analyte using said reaction product.

### CROSS-REFERENCE

This application is a continuation of International Patent Application No. PCT/US25/48840, filed Sep. 30, 2025, which claims the benefit and priority of U.S. Provisional Application Nos. 63/701,846 filed Oct. 1, 2024, 63/764,444 filed Feb. 27, 2025, and 63/793,208 filed Apr. 23, 2025, each of which applications are incorporated herein by reference for all purposes in its entirety.

### BACKGROUND

Proximity Ligation Assay (PLA) is an immunoassay method that integrates the specificity of antibody detection with PCR-based signal amplification. The use of PCR polymerase in PLA may have advantages. PCR polymerase may provide exponential amplification of the target signal, higher sensitivity, enabling the detection of minute quantities of the target protein. The specificity of PLA may be enhanced by dual antibody binding, which reduces the likelihood of false positives. However, amplification of background noise from PCR polymerase may impact sensitivity, and the non-specific binding of probes may be amplified and lead to false positives or an overestimation of the target protein's abundance.

### SUMMARY

In one aspect, the present disclosure provides a method for analyte detection, comprising: (a) providing a complex coupled to a surface of a substrate, wherein the complex comprises: (i) an analyte; (ii) a first probe coupled to the analyte; (iii) a second probe coupled to the analyte, wherein the first probe or the second probe is coupled to the surface of the substrate; (b) using the first probe and the second probe to generate a reaction product; and (c) detecting the analyte using the reaction product, with a detection limit of about 0.1 fg/mL or less for detecting the analyte, wherein (a)-(c) are performed in no more than about 90 minutes.

In some embodiments, the method further comprises, prior to (b), washing the complex coupled to the surface of the substrate at most once. In some embodiments, (a)-(c) are performed in no more than about 60 minutes. In some embodiments, (a)-(c) are performed in no more than about 30 minutes. In some embodiments, in (a), a density of the first probe or the second probe coupled to the surface of the substrate corresponds to no more than about 5% surface area occupancy of the surface. In some embodiments, the first probe or the second probe is coupled to the surface of the substrate via a binding pair. In some embodiments, the binding pair comprises biotin-streptavidin. In some embodiments, the surface is selected from the group consisting of a solid surface, a bead, a micropost, a microfluidic element, and a coated well. In some embodiments, the first probe or the second probe comprises an antibody. In some embodiments, the first probe or the second probe comprises an aptamer. In some embodiments, the first probe or the second probe comprises an antigen. In some embodiments, the analyte is a biomolecule. In some embodiments, the biomolecule is a protein. In some embodiments, in (b), the reaction product comprises a fluorescence resonance energy transfer (FRET) donor or acceptor.

In some embodiments, the method further comprises detecting the analyte using the reaction product with a specificity of greater than about 75%. In some embodiments, (i) the first probe comprises a first binding moiety coupled to a first biomolecule; and (ii) the second probe comprises a second binding moiety coupled to a second biomolecule. In some embodiments, (b) comprises reacting the first biomolecule with the second biomolecule to couple the first biomolecule to the second biomolecule. In some embodiments, the first biomolecule is a first nucleic acid molecule and the second biomolecule is a second nucleic acid molecule. In some embodiments, (b) comprises ligating a first element of the first nucleic acid molecule to a second element of the second nucleic acid molecule thereby generating a ligated nucleic acid molecule, wherein the ligated nucleic acid molecule couples the first probe to the second probe. In some embodiments, the method further comprises generating a complementary nucleic acid molecule of the ligated nucleic acid molecule. In some embodiments, the method further comprises amplifying the complementary nucleic acid molecule of the ligated nucleic acid molecule, thereby generating the reaction product. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction.

In some embodiments, (c) comprises sequencing the reaction product. In some embodiments, the substrate is fluorescently labeled.

In another aspect, the present disclosure provides a method for analyte detection, comprising: (a) providing: (i) a first probe coupled to a surface of a substrate, and (ii) the analyte; (b) binding the first probe coupled to the surface of the substrate and a second probe to the analyte, to generate a complex; (c) using the first probe and the second probe of the complex to generate a reaction product; and (d) detecting the analyte using the reaction product.

In some embodiments, in (c), the complex is coupled to the surface of the substrate. In some embodiments, the method further comprises, in (d), detecting the analyte using the reaction product with a detection limit of about 0.1 fg/mL or less for detecting the analyte. In some embodiments, (a)-(d) are performed in no more than about 90 minutes. In some embodiments, (a)-(d) are performed in no more than about 60 minutes. In some embodiments, (a)-(d) are performed in no more than about 30 minutes. In some embodiments, the method further comprises, prior to (c), washing the complex at most once. In some embodiments, in (a), a density of the first probe coupled to the surface of the substrate corresponds to no more than about 5% surface area occupancy of the surface. In some embodiments, the first probe is coupled to the surface of the substrate via a binding pair. In some embodiments, the binding pair comprise biotin-streptavidin. In some embodiments, the surface is selected from the group consisting of a solid surface, a bead, a micropost, a microfluidic element, and a coated well. In some embodiments, the first probe or the second probe comprises an antibody. In some embodiments, the first probe or the second probe comprises an aptamer. In some embodiments, the first probe or the second probe comprises an antigen. In some embodiments, the analyte is a biomolecule. In some embodiments, the biomolecule is a protein. In some embodiments, in (c), the reaction product comprises a fluorescence resonance energy transfer (FRET) donor or acceptor.

In some embodiments, the method further comprises detecting the analyte using the reaction product with a specificity of greater than about 75%. In some embodiments, (i) the first probe comprises a first binding moiety coupled to a first biomolecule; and (ii) the second probe comprises a second binding moiety coupled to a second biomolecule. In some embodiments, (c) comprises reacting the first biomolecule with the second biomolecule to couple the first biomolecule to the second biomolecule. In some embodiments, the first biomolecule is a first nucleic acid molecule and the second biomolecule is a second nucleic acid molecule. In some embodiments, (c) comprises ligating a first element of the first nucleic acid molecule to a second element of the second nucleic acid molecule thereby generating a ligated nucleic acid molecule, wherein the ligated nucleic acid molecule couples the first probe to the second probe. In some embodiments, the method further comprises generating a complementary nucleic acid molecule of the ligated nucleic acid molecule. In some embodiments, the method further comprises amplifying the complementary nucleic acid molecule of the ligated nucleic acid molecule, thereby generating the reaction product. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction.

In some embodiments, (d) comprises sequencing the reaction product. In some embodiments, the substrate is fluorescently labeled.

In another aspect, the present disclosure provides a method for analyte detection, comprising: (a) providing a complex coupled to a surface of a substrate, wherein the complex comprises: (i) an analyte; (ii) a first probe coupled to the analyte; (iii) a second probe coupled to the analyte, wherein the first probe or the second probe is coupled to the surface of the substrate; (b) using the first probe and the second probe to generate a reaction product; and (c) detecting the analyte using the reaction product, wherein, prior to (b), the complex coupled to the surface of the substrate is washed at most once.

In some embodiments, the method further comprises, in (c), detecting the analyte using the reaction product with a detection limit of about 0.1 fg/mL or less for detecting the analyte. In some embodiments, (a)-(c) are performed in no more than about 90 minutes. In some embodiments, (a)-(c) are performed in no more than about 60 minutes. In some embodiments, (a)-(c) are performed in no more than about 30 minutes. In some embodiments, in (a), a density of the first probe or the second probe coupled to the surface of the substrate corresponds to no more than about 5% surface area occupancy of the surface. In some embodiments, the first probe or the second probe is coupled to the surface of the substrate via a binding pair. In some embodiments, the binding pair comprises biotin-streptavidin. In some embodiments, the surface is selected from the group consisting of a solid surface, a bead, a micropost, a microfluidic element, and a coated well. In some embodiments, the first probe or the second probe comprises an antibody. In some embodiments, the first probe or the second probe comprises an aptamer. In some embodiments, the first probe or the second probe comprises an antigen. In some embodiments, the analyte is a biomolecule. In some embodiments, the biomolecule is a protein. In some embodiments, in (b), the reaction product comprises a fluorescence resonance energy transfer (FRET) donor or acceptor.

In some embodiments, the method further comprises detecting the analyte using the reaction product with a specificity of greater than about 75%. In some embodiments, in (b), the complex is coupled to the surface of the substrate. In some embodiments, (i) the first probe comprises a first binding moiety coupled to a first biomolecule; and (ii) the second probe comprises a second binding moiety coupled to a second biomolecule. In some embodiments, (b) comprises reacting the first biomolecule with the second biomolecule to couple the first biomolecule to the second biomolecule. 19. In some embodiments, the first biomolecule is a first nucleic acid molecule and the second biomolecule is a second nucleic acid molecule. In some embodiments, (b) comprises ligating a first element of the first nucleic acid molecule to a second element of the second nucleic acid molecule thereby generating a ligated nucleic acid molecule, wherein the ligated nucleic acid molecule couples the first probe to the second probe. In some embodiments, the method further comprises generating a complementary nucleic acid molecule of the ligated nucleic acid molecule. In some embodiments, the method further comprises amplifying the complementary nucleic acid molecule of the ligated nucleic acid molecule, thereby generating the reaction product. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction.

In some embodiments, (c) comprises sequencing the reaction product. In some embodiments, the substrate is fluorescently labeled.

In another aspect, the present disclosure provides a method for analyte detection, comprising: (a) generating a complex, wherein the complex comprises: (i) an analyte; (ii) a first probe coupled to the analyte; and (iii) a second probe coupled to the analyte; (b) without washing the complex after (a), coupling the complex generated in (a) to a surface of a substrate; (c) using the first probe and the second probe of the complex coupled to the surface of the substrate to generate a reaction product; and (d) detecting the analyte using the reaction product.

In some embodiments, the method further comprises, in (d), detecting the analyte using the reaction product with a detection limit of about 0.1 fg/mL or less for detecting the analyte. In some embodiments, (a)-(d) are performed in no more than about 90 minutes. In some embodiments, (a)-(d) are performed in no more than about 60 minutes. In some embodiments, wherein (a)-(d) are performed in no more than about 30 minutes. In some embodiments, the method further comprises, prior to (c), washing the complex coupled to the surface of the substrate at most once. In some embodiments, (i) the first probe or the second probe is coupled to the surface of the substrate; and (ii) a density of the first probe or the second probe coupled to the surface of the substrate corresponds to no more than about 5% surface area occupancy of the surface. In some embodiments, (i) the first probe or the second probe is coupled to the surface of the substrate; and (ii) the first probe or the second probe is coupled to the surface of the substrate via a binding pair. In some embodiments, the binding pair comprises biotin-streptavidin. In some embodiments, the surface is selected from the group consisting of a solid surface, a bead, a micropost, a microfluidic element, and a coated well. In some embodiments, the first probe or the second probe comprises an antibody. In some embodiments, the first probe or the second probe comprises an aptamer. In some embodiments, the first probe or the second probe comprises an antigen. In some embodiments, the analyte is a biomolecule. In some embodiments, the biomolecule is a protein. In some embodiments, in (c), the reaction product comprises a fluorescence resonance energy transfer (FRET) donor or acceptor.

In some embodiments, the method further comprises detecting the analyte using the reaction product with a specificity of greater than about 75%. In some embodiments, (i) the first probe comprises a first binding moiety coupled to a first biomolecule; and (ii) the second probe comprises a second binding moiety coupled to a second biomolecule. In some embodiments, (c) comprises reacting the first biomolecule with the second biomolecule to couple the first biomolecule to the second biomolecule. In some embodiments, the first biomolecule is a first nucleic acid molecule and the second biomolecule is a second nucleic acid molecule. In some embodiments, (c) comprises ligating a first element of the first nucleic acid molecule to a second element of the second nucleic acid molecule thereby generating a ligated nucleic acid molecule, wherein the ligated nucleic acid molecule couples the first probe to the second probe. In some embodiments, the method further comprises generating a complementary nucleic acid molecule of the ligated nucleic acid molecule. In some embodiments, the method further comprises amplifying the complementary nucleic acid molecule of the ligated nucleic acid molecule, thereby generating the reaction product. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction.

In some embodiments, (d) comprises sequencing the reaction product. In some embodiments, wherein the substrate is fluorescently labeled.

In another aspect, the present disclosure provides a composition, comprising a plurality of probes, wherein: (a) a first probe of the plurality of probes comprises: (1) a first binding moiety; and (2) a first biomolecule, wherein: (i) the first probe comprising the first biomolecule is coupled to a surface of a substrate, wherein a density of the first probe coupled to the surface of the substrate corresponds to no more than about 5% surface area occupancy of the surface; and (ii) the first binding moiety is configured to bind to an analyte; (b) a second probe of the plurality of probes comprises: (1) a second binding moiety; and (2) a second biomolecule, wherein the second binding moiety is configured to bind to the analyte.

In some embodiments, the first probe is coupled to the surface of the substrate via a binding pair. In some embodiments, the binding pair comprises biotin-streptavidin. In some embodiments, the surface is selected from the group consisting of a solid surface, a bead, a micropost, a microfluidic element, and a coated well. In some embodiments, the first binding moiety or the second binding moiety comprises an antibody. In some embodiments, the first binding moiety or the second binding moiety comprises an aptamer. In some embodiments, the first binding moiety or the second binding moiety comprises an antigen. In some embodiments, the analyte is a biomolecule. In some embodiments, the biomolecule is a protein. In some embodiments, the composition further comprises the analyte, wherein the first binding moiety and the second binding moiety are bound to the analyte. In some embodiments, the first biomolecule and the second biomolecule are positioned within sufficient distance to participate in a reaction with one another. In some embodiments, the first biomolecule and the second biomolecule are nucleic acid molecules. In some embodiments, the first binding moiety and the second binding moiety are antibodies. In some embodiments, the first biomolecule is a fluorescence resonance energy transfer (FRET) donor or acceptor. In some embodiments, the composition further comprises a ligase. In some embodiments, the substrate is fluorescently labeled.

In another aspect, the present disclosure provides a method of detecting an analyte, comprising: (a) providing a complex coupled to a surface of a substrate, the complex comprising: (A) a first probe comprising: (1) a first binding moiety; and (2) a first biomolecule, wherein: (i) the first probe comprises the first biomolecule and is coupled to the surface of the substrate; (ii) a density of the first probe coupled to the surface of the substrate corresponds to no more than about 5% surface area occupancy of the surface; and (iii) the first binding moiety is bound to an analyte; (B) a second probe comprising: (1) a second binding moiety; and (2) a second biomolecule, wherein the second binding moiety is bound to the analyte; (b) using the first probe and the second probe to generate a reaction product; and (c) detecting the analyte using the reaction product.

In some embodiments, the method further comprises, in (c), detecting the analyte using the reaction product with a detection limit of about 0.1 fg/mL or less for detecting the analyte. In some embodiments, the method further comprises, in (c), detecting the analyte using the reaction product with a specificity of greater than about 75%. In some embodiments, the method further comprises, prior to (b), washing the complex coupled to the surface of the substrate at most once. In some embodiments, (a)-(c) are performed in no more than about 90 minutes. In some embodiments, (a)-(c) are performed in no more than about 60 minutes. In some embodiments, (a)-(c) are performed in no more than about 30 minutes. In some embodiments, the first probe is coupled to the surface of the substrate via a binding pair. In some embodiments, the binding pair comprises biotin-streptavidin. In some embodiments, the surface is selected from the group consisting of a solid surface, a bead, a micropost, a microfluidic element, and a coated well. In some embodiments, the first binding moiety or the second binding moiety comprises an antibody. In some embodiments, the first binding moiety or the second binding moiety comprises an aptamer. In some embodiments, the first binding moiety or the second binding moiety comprises an antigen. In some embodiments, the analyte is a biomolecule. In some embodiments, the biomolecule is a protein. In some embodiments, in (b), the reaction product comprises a fluorescence resonance energy transfer (FRET) donor or acceptor.

In some embodiments, (b) comprises reacting the first biomolecule with the second biomolecule to couple the first biomolecule to the second biomolecule. In some embodiments, the first biomolecule is a first nucleic acid molecule and the second biomolecule is a second nucleic acid molecule. In some embodiments, wherein (b) comprises ligating a first element of the first nucleic acid molecule to a second element of the second nucleic acid molecule thereby generating a ligated nucleic acid molecule, wherein the ligated nucleic acid molecule couples the first probe to the second probe. In some embodiments, the method further comprises generating a complementary nucleic acid molecule of the ligated nucleic acid molecule. In some embodiments, the method further comprises amplifying the complementary nucleic acid molecule of the ligated nucleic acid molecule, thereby generating the reaction product. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction.

In some embodiments, (c) comprises sequencing the reaction product. In some embodiments, the substrate is fluorescently labeled.

In another aspect, the present disclosure provides a composition, comprising a plurality of probes, wherein: (a) a first probe of the plurality of probes comprises: (1) a first binding moiety covalently attached to a first member of a binding pair; and (2) a first nucleic acid molecule, wherein the first binding moiety is configured to bind to an analyte; (b) a surface of a substrate comprising a second member of the binding pair; and (c) a second probe of the plurality of probes comprises: (1) a second binding moiety; and (2) a second nucleic acid molecule, wherein the second binding moiety is configured to bind to the analyte.

In some embodiments, the first member of the binding pair is bound to the second member of the binding pair, such that the first probe is bound to the surface of the substrate. In some embodiments, a density of the first probe bound to the surface of the substrate corresponds to no more than about 5% surface area occupancy of the surface. In some embodiments, the binding pair comprises biotin-streptavidin. In some embodiments, the surface is selected from the group consisting of a solid surface, a bead, a micropost, a microfluidic element, and a coated well. In some embodiments, the first binding moiety or the second binding moiety comprises an antibody. In some embodiments, the first binding moiety or the second binding moiety an aptamer. In some embodiments, the first binding moiety or the second binding moiety comprises an antigen. In some embodiments, the analyte is a biomolecule. In some embodiments, the biomolecule is a protein. In some embodiments, the composition further comprises the analyte, wherein the first binding moiety and the second binding moiety are bound to the analyte. In some embodiments, the first nucleic acid molecule and the second nucleic acid molecule are positioned within sufficient distance to participate in a reaction with one another. In some embodiments, the first binding moiety and the second binding moiety are antibodies. In some embodiments, the composition further comprises a ligase. In some embodiments, the substrate is fluorescently labeled.

In another aspect, the present disclosure provides a method of detecting an analyte, comprising: (a) providing a complex on a surface of a substrate, the complex comprising: (A) a first probe comprising: (1) a first binding moiety covalently attached to a first member of a binding pair; and (2) a first nucleic acid molecule, wherein the first binding moiety is bound to an analyte; (B) the surface of the substrate comprising a second member of the binding pair, the second member of the binding pair bound to the first member of the binding pair such that the first probe is bound to the surface; and (C) a second probe comprising: (1) a second binding moiety; and (2) a second nucleic acid molecule, wherein the second binding moiety is configured to bind to the analyte; (b) using the first probe and the second probe to generate a reaction product; and (c) detecting the analyte using the reaction product.

In some embodiments, a density of the first probe bound to the surface of the substrate corresponds to no more than about 5% surface area occupancy of the surface. In some embodiments, the method further comprises, in (c), detecting the analyte using the reaction product with a detection limit of about 0.1 fg/mL or less for detecting the analyte. In some embodiments, the method further comprises, in (c), detecting the analyte using the reaction product with a specificity of greater than about 75%. In some embodiments, the method further comprises, prior to (b), washing the complex coupled to the surface of the substrate at most once. In some embodiments, (a)-(c) are performed in no more than about 90 minutes. In some embodiments, (a)-(c) are performed in no more than about 60 minutes. In some embodiments, (a)-(c) are performed in no more than about 30 minutes. In some embodiments, the binding pair comprises biotin-streptavidin. In some embodiments, the surface is selected from the group consisting of a solid surface, a bead, a micropost, a microfluidic element, and a coated well. In some embodiments, the first binding moiety or the second binding moiety comprises an antibody. In some embodiments, the first binding moiety or the second binding moiety comprises an aptamer. In some embodiments, the first binding moiety or the second binding moiety comprises an antigen. In some embodiments, the analyte is a biomolecule. In some embodiments, the biomolecule is a protein. In some embodiments, in (b), the reaction product comprises a fluorescence resonance energy transfer (FRET) donor or acceptor.

In some embodiments, (b) comprises reacting the first nucleic acid molecule with the second nucleic acid molecule to couple the first nucleic acid molecule to the second nucleic acid molecule. In some embodiments, (b) comprises ligating a first element of the first nucleic acid molecule to a second element of the second nucleic acid molecule thereby generating a ligated nucleic acid molecule, wherein the ligated nucleic acid molecule couples the first probe to the second probe. In some embodiments, the method further comprises generating a complementary nucleic acid molecule of the ligated nucleic acid molecule. In some embodiments, the method further comprises amplifying the complementary nucleic acid molecule of the ligated nucleic acid molecule, thereby generating the reaction product. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction.

In some embodiments, (c) comprises sequencing the reaction product. In some embodiments, the substrate is fluorescently labeled.

In another aspect, the present disclosure provides a composition, comprising a plurality of probes, wherein: (a) a first probe of the plurality of probes comprises: (1) a first binding moiety; and (2) a first biomolecule, wherein: (i) the first biomolecule is coupled to the first binding moiety; (ii) the first probe comprising the first biomolecule is positioned on a surface of a substrate, wherein a density of the first probe on the surface of the substrate corresponds to no more than about 5% surface area occupancy; and (iii) the first binding moiety is configured to bind to an analyte; (b) a second probe of the plurality of probes comprises: (1) a second binding moiety; and (2) a second biomolecule, wherein: (i) the second biomolecule is coupled to the second binding moiety; and (ii) the second binding moiety is configured to bind to the analyte.

In some embodiments, the first biomolecule identifies the first binding moiety. In some embodiments, the second biomolecule identifies the second binding moiety. In some embodiments, in (a), the first probe of the plurality of probes further comprises: (3) a first member of a binding pair, and (4) a second member of the binding pair. In some embodiments, (a) further comprises the first binding moiety is coupled to the first member of the binding pair. In some embodiments, (a) further comprises the second member of the binding pair is coupled to the surface of the substrate. In some embodiments, (a) further comprises the first member of the binding pair is coupled to the second member of the binding pair, positioning the first binding moiety at the surface of the substrate. In some embodiments, (a) further comprises the first biomolecule and the first member of the binding pair are coupled to the first binding moiety at different positions of the first binding moiety. In some embodiments, the first member of the binding pair comprises biotin. In some embodiments, the second member of the binding pair comprises streptavidin.

In some embodiments, the surface is a solid surface. In some embodiments, the surface comprises a bead. In some embodiments, the surface comprises a micropost. In some embodiments, the surface comprises a microfluidic element. In some embodiments, the surface comprises a coated well. In some embodiments, the first binding moiety or the second binding moiety is an antibody. In some embodiments, the first binding moiety or the second binding moiety is an aptamer. In some embodiments, the first binding moiety or the second binding moiety is an antigen. In some embodiments, the first binding moiety and the second binding moiety are antibodies. In some embodiments, the first binding moiety and the second binding moiety are aptamers. In some embodiments, the first binding moiety and the second binding moiety are antigens. In some embodiments, the analyte is an antibody.

In some embodiments, in (a)(iii), the first binding moiety is configured to bind to an epitope of the analyte. In some embodiments, in (b)(ii), the second binding moiety is configured to bind to a different epitope of the analyte.

In some embodiments, (b) further comprises (iii) the analyte bound to the first probe and the second probe, wherein the first biomolecule and the second biomolecule are positioned within sufficient distance to participate in a reaction with one another. In some embodiments, the reaction is a proximity ligation assay (PLA). In some embodiments, the reaction is a fluorescence resonance energy transfer (FRET) assay. In some embodiments, the reaction is a colocalization reaction. In some embodiments, the reaction has a sensitivity of greater than about 0.1 fg/mL. In some embodiments, the reaction has a specificity of greater than about 75%.

In another aspect, the present disclosure provides a composition, comprising a plurality of probes, wherein: (a) a first probe of the plurality of probes comprises: (1) a first binding moiety; (2) a first biomolecule; (3) a first member of a binding pair; and (4) a second member of the binding pair, wherein: (i) the first biomolecule is coupled to the first binding moiety; (ii) the first binding moiety is coupled to the first member of the binding pair; (iii) the second member of the binding pair is coupled to a surface of a substrate; (iv) the first member of the binding pair is coupled to the second member of the binding pair, positioning the first binding moiety at the surface of the substrate; (v) the first biomolecule and the first member of the binding pair are coupled to the first binding moiety at different positions of the first binding moiety; and (vi) the first binding moiety is configured to bind to an analyte; (b) a second probe of the plurality of probes comprises: (1) a second binding moiety; and (2) a second biomolecule, wherein: (i) the second biomolecule is coupled to the second binding moiety; and (ii) the second binding moiety is configured to bind to the analyte.

In some embodiments, the first biomolecule identifies the first binding moiety. In some embodiments, the second biomolecule identifies the second binding moiety. In some embodiments, a density of the first probe on the surface of the substrate corresponds to no more than about 5% surface area occupancy. In some embodiments, the first member of the binding pair comprises biotin. In some embodiments, the second member of the binding pair comprises streptavidin.

In some embodiments, the surface is a solid surface. In some embodiments, the surface comprises a bead. In some embodiments, the surface comprises a micropost. In some embodiments, the surface comprises a microfluidic element. In some embodiments, the surface comprises a coated well. In some embodiments, the first binding moiety or the second binding moiety is an antibody. In some embodiments, the first binding moiety or the second binding moiety is an aptamer. In some embodiments, the first binding moiety or the second binding moiety is an antigen. In some embodiments, the first binding moiety and the second binding moiety are antibodies. In some embodiments, the first binding moiety and the second binding moiety are aptamers. In some embodiments, the first binding moiety and the second binding moiety are antigens. In some embodiments, the analyte is an antibody.

In some embodiments, in (a)(vi), the first binding moiety is configured to bind to an epitope of the analyte. In some embodiments, in (b)(ii), the second binding moiety is configured to bind to a different epitope of the analyte.

In some embodiments, (b) further comprises (iii) the analyte bound to the first probe and the second probe, wherein the first biomolecule and the second biomolecule are positioned within sufficient distance to participate in a reaction with one another. In some embodiments, the reaction is a proximity ligation assay (PLA). In some embodiments, the reaction is a fluorescence resonance energy transfer (FRET) assay. In some embodiments, the reaction is a colocalization reaction. In some embodiments, the reaction has a sensitivity of greater than about 0.1 fg/mL. In some embodiments, the reaction has a specificity of greater than about 75%.

In another aspect, the present disclosure provides a method for detecting an analyte, comprising: (a) providing: (1) a plurality of probes, comprising a first probe and a second probe; (2) the first probe of the plurality of probes comprises a first binding moiety and a first biomolecule, wherein: (i) the first biomolecule is coupled to the first binding moiety; (ii) the first probe comprising the first biomolecule is positioned on a surface of a substrate, wherein a density of the first probe on the surface of the substrate corresponds to no more than about 5% surface area occupancy; and (iii) the first binding moiety is configured to bind to the analyte; (3) the second probe of the plurality of probes comprises a second binding moiety and a second biomolecule, wherein: (i) the second biomolecule is coupled to the second binding moiety; and (ii) the second binding moiety is configured to bind to the analyte; (b) detecting the first biomolecule and the second biomolecule, thereby detecting the analyte.

In some embodiments, in (a)(2), the providing the first probe of the plurality of probes further comprises a first member of a binding pair, and a second member of the binding pair. In some embodiments, (a)(2) further comprises the first binding moiety is coupled to the first member of the binding pair. In some embodiments, (a)(2) further comprises the second member of the binding pair is coupled to the surface of the substrate. In some embodiments, (a)(2) further comprises the first member of the binding pair is coupled to the second member of the binding pair, positioning the first binding moiety at the surface of the substrate. In some embodiments, (a)(2) further comprises the first biomolecule and the first member of the binding pair are coupled to the first binding moiety at different positions of the first binding moiety. In some embodiments, the first member of the binding pair comprises biotin. In some embodiments, the second member of the binding pair comprises streptavidin.

In some embodiments, the surface is a solid surface. In some embodiments, the surface comprises a bead. In some embodiments, the surface comprises a micropost. In some embodiments, the surface comprises a microfluidic element. In some embodiments, the surface comprises a coated well. In some embodiments, the first binding moiety or the second binding moiety is an antibody. In some embodiments, the first binding moiety or the second binding moiety is an aptamer. In some embodiments, the first binding moiety or the second binding moiety is an antigen. In some embodiments, the first binding moiety and the second binding moiety are antibodies. In some embodiments, the first binding moiety and the second binding moiety are aptamers. In some embodiments, the first binding moiety and the second binding moiety are antigens. In some embodiments, the analyte is an antibody.

In some embodiments, in (a)(2)(iii), the first binding moiety is configured to bind to an epitope of the analyte. In some embodiments, in (a)(3)(ii), the second binding moiety is configured to bind to a different epitope of the analyte.

In some embodiments, (a)(3) further comprises (iii) the analyte bound to the first probe and the second probe, wherein the first biomolecule and the second biomolecule are positioned within sufficient distance to participate in a reaction with one another. In some embodiments, the reaction is a proximity ligation assay (PLA). In some embodiments, the reaction is a fluorescence resonance energy transfer (FRET) assay. In some embodiments, the reaction is a colocalization reaction. In some embodiments, the reaction has a sensitivity of greater than about 0.1 fg/mL. In some embodiments, the reaction has a specificity of greater than about 75%.

In some embodiments, the method further comprises (c) ligating the first element of the first biomolecule and the second element of the second biomolecule using a ligase, thereby generating a ligated nucleic acid template. In some embodiments, the method further comprises comprising (d) generating a complementary sequence of the ligated nucleic acid template.

In some embodiments, in (d), the generating the complementary sequence of the ligated nucleic acid template comprises thermocycling the ligated nucleic acid template. In some embodiments, in (d), the complementary sequence of the ligated nucleic acid template is positioned on the surface of the substrate. In some embodiments, in (d), the complementary sequence of the ligated nucleic acid template is released from the surface of the substrate. In some embodiments, the thermocycling comprises inactivating the ligase. In some embodiments, the thermocycling comprises using a polymerase.

In some embodiments, the method further comprises (e) amplifying the complementary sequence of the ligated nucleic acid template, thereby generating a nucleic acid molecule. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction. In some embodiments, the method further comprises (f) sequencing the nucleic acid molecule. In some embodiments, the substrate is fluorescently labeled. In some embodiments, the sequencing is conducted on the fluorescently labeled substrate.

In another aspect, the present disclosure provides a method for detecting an analyte, comprising: (a) providing: (1) a plurality of probes, comprising a first probe and a second probe; (2) the first probe of the plurality of probes comprises a first binding moiety and a first biomolecule, wherein: (i) the first biomolecule is coupled to the first binding moiety; (ii) the first probe comprising the first biomolecule is positioned on a surface of a substrate, wherein a density of the first probe on the surface of the substrate corresponds to no more than about 5% surface area occupancy; and (iii) the first binding moiety is configured to bind to the analyte; (3) the second probe of the plurality of probes comprises a second binding moiety and a second biomolecule, wherein: (i) the second biomolecule is coupled to the second binding moiety; and (ii) the second binding moiety is configured to bind to the analyte; (b) conducting a reaction between a first element of the first biomolecule and a second element of the second biomolecule.

In some embodiments, the first biomolecule identifies the first binding moiety. In some embodiments, the second biomolecule identifies the second binding moiety. In some embodiments, in (a)(2), the providing the first probe of the plurality of probes further comprises a first member of a binding pair, and a second member of the binding pair. In some embodiments, (a)(2) further comprises the first binding moiety is coupled to the first member of the binding pair. In some embodiments, (a)(2) further comprises the second member of the binding pair is coupled to the surface of the substrate. In some embodiments, (a)(2) further comprises the first member of the binding pair is coupled to the second member of the binding pair, positioning the first binding moiety at the surface of the substrate. In some embodiments, (a)(2) further comprises the first biomolecule and the first member of the binding pair are coupled to the first binding moiety at different positions of the first binding moiety. In some embodiments, the first member of the binding pair comprises biotin. In some embodiments, the second member of the binding pair comprises streptavidin.

In some embodiments, the surface is a solid surface. In some embodiments, the surface comprises a bead. In some embodiments, the surface comprises a micropost. In some embodiments, the surface comprises a microfluidic element. In some embodiments, the surface comprises a coated well. In some embodiments, the first binding moiety or the second binding moiety is an antibody. In some embodiments, the first binding moiety or the second binding moiety is an aptamer. In some embodiments, the first binding moiety or the second binding moiety is an antigen. In some embodiments, the first binding moiety and the second binding moiety are antibodies. In some embodiments, the first binding moiety and the second binding moiety are aptamers. In some embodiments, the first binding moiety and the second binding moiety are antigens. In some embodiments, the analyte is an antibody.

In some embodiments, in (a)(2)(iii), the first binding moiety is configured to bind to an epitope of the analyte. In some embodiments, in (a)(3)(ii), the second binding moiety is configured to bind to a different epitope of the analyte.

In some embodiments, in (b), the first element of the first biomolecule and the second element of the second biomolecule are positioned within sufficient distance to participate in the reaction. In some embodiments, the reaction is a proximity ligation assay (PLA). In some embodiments, the reaction is a fluorescence resonance energy transfer (FRET) assay. In some embodiments, the reaction is a colocalization reaction. In some embodiments, the reaction has a sensitivity of greater than about 0.1 fg/mL. In some embodiments, the reaction has a specificity of greater than about 75%.

In some embodiments, the method further comprises (c) ligating the first element of the first biomolecule and the second element of the second biomolecule using a ligase, thereby generating a ligated nucleic acid template across the first probe and the second probe. In some embodiments, the method further comprises (d) generating a complementary sequence of the ligated nucleic acid template.

In some embodiments, in (d), the generating the complementary sequence of the ligated nucleic acid template comprises thermocycling the ligated nucleic acid template. In some embodiments, in (d), the complementary sequence of the ligated nucleic acid template is positioned on the surface of the substrate. In some embodiments, in (d), the complementary sequence of the ligated nucleic acid template is released from the surface of the substrate. In some embodiments, the thermocycling comprises inactivating the ligase. In some embodiments, the thermocycling comprises using a polymerase.

In some embodiments, the method further comprises (e) amplifying the complementary sequence of the ligated nucleic acid template, thereby generating a nucleic acid molecule. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction. In some embodiments, the method further comprises (f) sequencing the nucleic acid molecule. In some embodiments, the substrate is fluorescently labeled. In some embodiments, the sequencing is conducted on the fluorescently labeled substrate.

In another aspect, the present disclosure provides a method of detecting an analyte, comprising: (a) providing: (1) a plurality of probes, comprising a first probe and a second probe, (2) the first probe of the plurality of probes comprising a first binding moiety, a first biomolecule, a first member of a binding pair, and a second member of the binding pair, wherein: (i) the first biomolecule is coupled to the first binding moiety; (ii) the first binding moiety is coupled to the first member of the binding pair; (iii) the second member of the binding pair is coupled to a surface of a substrate; (iv) the first member of the binding pair is coupled to the second member of the binding pair, positioning the first binding moiety at the surface of the substrate; (v) the first biomolecule and the first member of the binding pair are coupled to the first binding moiety at different positions of the first binding moiety; and (vi) the first binding moiety is configured to bind to the analyte; (3) the second probe of the plurality of probes comprising: a second binding moiety, and a second biomolecule, wherein: (i) the second biomolecule is coupled to the second binding moiety; (ii) the second binding moiety is configured to bind to the analyte; and (iii) the analyte bound to the first probe and the second probe; (b) detecting the first biomolecule and the second biomolecule, thereby detecting the analyte.

In some embodiments, a density of the first probe on the surface of the substrate corresponds to no more than about 5% surface area occupancy. In some embodiments, the first member of the binding pair comprises biotin. In some embodiments, the second member of the binding pair comprises streptavidin.

In some embodiments, the surface is a solid surface. In some embodiments, the surface comprises a bead. In some embodiments, the surface comprises a micropost. In some embodiments, the surface comprises a microfluidic element. In some embodiments, the surface comprises a coated well. In some embodiments, the first binding moiety or the second binding moiety is an antibody. In some embodiments, the first binding moiety or the second binding moiety is an aptamer. In some embodiments, the first binding moiety or the second binding moiety is an antigen. In some embodiments, the first binding moiety and the second binding moiety are antibodies. In some embodiments, the first binding moiety and the second binding moiety are aptamers. In some embodiments, the first binding moiety and the second binding moiety are antigens. In some embodiments, the analyte is an antibody.

In some embodiments, in (a)(2)(vi), the first binding moiety is configured to bind to an epitope of the analyte. In some embodiments, in (a)(3)(ii), the second binding moiety is configured to bind to a different epitope of the analyte.

In some embodiments, in (a)(3)(iii), the first biomolecule and the second biomolecule are positioned within sufficient distance to participate in a reaction with one another. In some embodiments, the reaction is a proximity ligation assay (PLA). In some embodiments, the reaction is a fluorescence resonance energy transfer (FRET) assay. In some embodiments, the reaction is a colocalization reaction. In some embodiments, the reaction has a sensitivity of greater than about 0.1 fg/mL. In some embodiments, the reaction has a specificity of greater than about 75%.

In some embodiments, the method further comprises (c) ligating the first element of the first biomolecule and the second element of the second biomolecule using a ligase, thereby generating a ligated nucleic acid template across the first probe and the second probe. In some embodiments, the method further comprises (d) generating a complementary sequence of the ligated nucleic acid template.

In some embodiments, in (d), the generating the complementary sequence of the ligated nucleic acid template comprises thermocycling the ligated nucleic acid template. In some embodiments, in (d), the complementary sequence of the ligated nucleic acid template is positioned on the surface of the substrate. In some embodiments, in (d), the complementary sequence of the ligated nucleic acid template is released from the surface of the substrate. In some embodiments, the thermocycling comprises inactivating the ligase. In some embodiments, the thermocycling comprises using a polymerase.

In some embodiments, the method further comprises (e) amplifying the complementary sequence of the ligated nucleic acid template, thereby generating a nucleic acid molecule. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction. In some embodiments, the method further comprises (f) sequencing the nucleic acid molecule. In some embodiments, the substrate is fluorescently labeled. In some embodiments, the sequencing is conducted on the fluorescently labeled substrate.

In another aspect, the present disclosure provides a method of detecting an analyte, comprising: (a) providing: (1) a plurality of probes, comprising a first probe and a second probe, (2) the first probe of the plurality of probes comprising a first binding moiety, a first biomolecule, a first member of a binding pair, and a second member of the binding pair, wherein: (i) the first biomolecule is coupled to the first binding moiety; (ii) the first binding moiety is coupled to the first member of the binding pair; (iii) the second member of the binding pair is coupled to a surface of a substrate; (iv) the first member of the binding pair is coupled to the second member of the binding pair, positioning the first binding moiety at the surface of the substrate; (v) the first biomolecule and the first member of the binding pair are coupled to the first binding moiety at different positions of the first binding moiety; and (vi) the first binding moiety is configured to bind to an analyte; (3) the second probe of the plurality of probes comprising: a second binding moiety, and a second biomolecule, wherein: (i) the second biomolecule is coupled to the second binding moiety; (ii) the second binding moiety is configured to bind to the analyte; and (iii) the analyte bound to the first probe and the second probe; (b) conducting a reaction between an element of the first biomolecule and an element of the second biomolecule.

In some embodiments, the first biomolecule identifies the first binding moiety. In some embodiments, the second biomolecule identifies the second binding moiety. In some embodiments, a density of the first probe on the surface of the substrate corresponds to no more than about 5% surface area occupancy. In some embodiments, the first member of the binding pair comprises biotin. In some embodiments, the second member of the binding pair comprises streptavidin.

In some embodiments, the surface is a solid surface. In some embodiments, the surface comprises a bead. In some embodiments, the surface comprises a micropost. In some embodiments, the surface comprises a microfluidic element. In some embodiments, the surface comprises a coated well. In some embodiments, the first binding moiety or the second binding moiety is an antibody. In some embodiments, the first binding moiety or the second binding moiety is an aptamer. In some embodiments, the first binding moiety or the second binding moiety is an antigen. In some embodiments, the first binding moiety and the second binding moiety are antibodies. In some embodiments, the first binding moiety and the second binding moiety are aptamers. In some embodiments, the first binding moiety and the second binding moiety are antigens. In some embodiments, the analyte is an antibody.

In some embodiments, in (a)(2)(vi), the first binding moiety is configured to bind to an epitope of the analyte. In some embodiments, in (a)(3)(ii), the second binding moiety is configured to bind to a different epitope of the analyte.

In some embodiments, in (b), the first element of the first biomolecule and the second element of the second biomolecule are positioned within sufficient distance to participate in the reaction. In some embodiments, the reaction is a proximity ligation assay (PLA). In some embodiments, the reaction is a fluorescence resonance energy transfer (FRET) assay. In some embodiments, the reaction is a colocalization reaction. In some embodiments, the reaction has a sensitivity of greater than about 0.1 fg/mL. In some embodiments, the reaction has a specificity of greater than about 75%.

In some embodiments, the method further comprises (c) ligating the first element of the first biomolecule and the second element of the second biomolecule using a ligase, thereby generating a ligated nucleic acid template. In some embodiments, the method further comprises (d) generating a complementary sequence of the ligated nucleic acid template.

In some embodiments, in (d), the generating the complementary sequence of the ligated nucleic acid template comprises thermocycling the ligated nucleic acid template. In some embodiments, in (d), the complementary sequence of the ligated nucleic acid template is positioned on the surface of the substrate. In some embodiments, in (d), the complementary sequence of the ligated nucleic acid template is released from the surface of the substrate. In some embodiments, the thermocycling comprises inactivating the ligase. In some embodiments, the thermocycling comprises using a polymerase.

In some embodiments, the method further comprises (e) amplifying the complementary sequence of the ligated nucleic acid template, thereby generating a nucleic acid molecule. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction. In some embodiments, the method further comprises (f) sequencing the nucleic acid molecule. In some embodiments, the substrate is fluorescently labeled. In some embodiments, the sequencing is conducted on the fluorescently labeled substrate.

In another aspect, the present disclosure provides a method for detecting an analyte, comprising: (a) binding a plurality of probes, comprising a first probe and a second probe, to the analyte, wherein: (1) the first probe of the plurality of probes is configured to bind to the analyte; (2) the first probe is positioned on a surface of a substrate; and (3) the second probe of the plurality of probes is configured to bind to the analyte; (b) conducting a reaction between the first probe and the second probe with a sensitivity of greater than about 0.1 fg/mL for identifying the analyte.

In some embodiments, in (a)(1), the first probe of the plurality of probes, further comprises a first binding moiety and a first biomolecule. In some embodiments, the first biomolecule is coupled to the first binding moiety. In some embodiments, in (a)(2), the first probe comprises the first biomolecule is positioned on the surface of the substrate. In some embodiments, in (a)(2), a density of the first probe on the surface of the substrate corresponds to no more than about 5% surface area occupancy. In some embodiments, in (a)(1), the first binding moiety of the first probe of the plurality of probes is configured to bind to the analyte. In some embodiments, in (a)(3), the second probe of the plurality of probes, further comprises a second binding moiety and a second biomolecule. In some embodiments, the second biomolecule is coupled to the second binding moiety. In some embodiments, in (a)(3), the second binding moiety of the second probe of the plurality of probes is configured to bind to the analyte. In some embodiments, the first biomolecule identifies the first binding moiety. In some embodiments, the second biomolecule identifies the second binding moiety. In some embodiments, in (a)(1), the first probe of the plurality of probes, further comprises a first member of a binding pair, and a second member of the binding pair.

In some embodiments, (a)(1) further comprises the first binding moiety is coupled to the first member of the binding pair. In some embodiments, (a)(2) further comprises the second member of the binding pair is coupled to the surface of the substrate. In some embodiments, (a)(2) further comprises the first member of the binding pair is coupled to the second member of the binding pair, positioning the first binding moiety at the surface of the substrate. In some embodiments, (a)(2) further comprises the first biomolecule and the first member of the binding pair are coupled to the first binding moiety at different positions of the first binding moiety. In some embodiments, the first member of the binding pair comprises biotin. In some embodiments, the second member of the binding pair comprises streptavidin.

In some embodiments, the surface is a solid surface. In some embodiments, the surface comprises a bead. In some embodiments, the surface comprises a micropost. In some embodiments, the surface comprises a microfluidic element. In some embodiments, the surface comprises a coated well. In some embodiments, the first binding moiety or the second binding moiety is an antibody. In some embodiments, the first binding moiety or the second binding moiety is an aptamer. In some embodiments, the first binding moiety or the second binding moiety is an antigen. In some embodiments, the first binding moiety and the second binding moiety are antibodies. In some embodiments, the first binding moiety and the second binding moiety are aptamers. In some embodiments, the first binding moiety and the second binding moiety are antigens. In some embodiments, the analyte is an antibody.

In some embodiments, the first binding moiety is configured to bind to an epitope of the analyte. In some embodiments, the second binding moiety is configured to bind to a different epitope of the analyte.

In some embodiments, in (b), the first probe and the second probe are positioned within sufficient distance to participate in the reaction with one another. In some embodiments, the reaction is a proximity ligation assay (PLA). In some embodiments, the reaction is a fluorescence resonance energy transfer (FRET) assay. In some embodiments, the reaction is a colocalization reaction. In some embodiments, the reaction has a sensitivity of greater than about 0.1 fg/mL. In some embodiments, the reaction has a specificity of greater than about 75%.

In some embodiments, the method further comprises (c) ligating a first element of the first biomolecule and a second element of the second biomolecule using a ligase, thereby generating a ligated nucleic acid template across the first probe and the second probe. In some embodiments, the method further comprises (d) generating a complementary sequence of the ligated nucleic acid template.

In some embodiments, in (d), the generating the complementary sequence of the ligated nucleic acid template comprises thermocycling the ligated nucleic acid template. In some embodiments, in (d), the complementary sequence of the ligated nucleic acid template is positioned on the surface of the substrate. In some embodiments, in (d), the complementary sequence of the ligated nucleic acid template is released from the surface of the substrate. In some embodiments, the thermocycling comprises inactivating the ligase. In some embodiments, the thermocycling comprises using a polymerase.

In some embodiments, the method further comprises (e) amplifying the complementary sequence of the ligated nucleic acid template, thereby generating a nucleic acid molecule. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction. In some embodiments, the method further comprises (f) sequencing the nucleic acid molecule. In some embodiments, the substrate is fluorescently labeled. In some embodiments, the sequencing is conducted on the fluorescently labeled substrate.

In another aspect, the present disclosure provides a method for detecting an analyte, comprising: (a) binding a plurality of probes, comprising a first probe and a second probe, to the analyte, wherein: (1) the first probe of the plurality of probes is configured to bind to the analyte; and (2) the second probe of the plurality of probes is configured to bind to the analyte, wherein the first probe and the second probe are positioned within sufficient distance to participate in a reaction with one another; and (b) conducting the reaction between the first probe and the second probe with a sensitivity of greater than about 0.1 fg/mL for identifying the analyte.

In some embodiments, in (a)(1), the first probe of the plurality of probes, further comprises a first binding moiety and a first biomolecule. In some embodiments, the first biomolecule is coupled to the first binding moiety. In some embodiments, in (a)(1), the first probe further comprises the first biomolecule is positioned on a surface of a substrate. In some embodiments, a density of the first probe on the surface of the substrate corresponds to no more than about 5% surface area occupancy. In some embodiments, in (a)(1), the first binding moiety of the first probe of the plurality of probes is configured to bind to the analyte. In some embodiments, in (a)(2), the second probe of the plurality of probes, further comprises a second binding moiety and a second biomolecule. In some embodiments, the second biomolecule is coupled to the second binding moiety. In some embodiments, in (a)(2), the second binding moiety of the second probe of the plurality of probes is configured to bind to the analyte.

In some embodiments, the first biomolecule identifies the first binding moiety. In some embodiments, the second biomolecule identifies the second binding moiety. In some embodiments, in (a)(1), the first probe of the plurality of probes, further comprises a first member of a binding pair, and a second member of the binding pair. In some embodiments, (a)(1) further comprises the first binding moiety is coupled to the first member of the binding pair. In some embodiments, (a)(1) further comprises the second member of the binding pair is coupled to the surface of the substrate. In some embodiments, (a)(1) further comprises the first member of the binding pair is coupled to the second member of the binding pair, positioning the first binding moiety at the surface of the substrate. In some embodiments, (a)(1) further comprises the first biomolecule and the first member of the binding pair are coupled to the first binding moiety at different positions of the first binding moiety. In some embodiments, the first member of the binding pair comprises biotin. In some embodiments, the second member of the binding pair comprises streptavidin.

In some embodiments, the surface is a solid surface. In some embodiments, the surface comprises a bead. In some embodiments, the surface comprises a micropost. In some embodiments, the surface comprises a microfluidic element. In some embodiments, the surface comprises a coated well. In some embodiments, the first binding moiety or the second binding moiety is an antibody. In some embodiments, the first binding moiety or the second binding moiety is an aptamer. In some embodiments, the first binding moiety or the second binding moiety is an antigen. In some embodiments, the first binding moiety and the second binding moiety are antibodies. In some embodiments, the first binding moiety and the second binding moiety are aptamers. In some embodiments, the first binding moiety and the second binding moiety are antigens. In some embodiments, the analyte is an antibody.

In some embodiments, the first binding moiety is configured to bind to an epitope of the analyte. In some embodiments, the second binding moiety is configured to bind to a different epitope of the analyte.

In some embodiments, the reaction is a proximity ligation assay (PLA). In some embodiments, the reaction is a fluorescence resonance energy transfer (FRET) assay. In some embodiments, the reaction is a colocalization reaction. In some embodiments, the reaction has a sensitivity of greater than about 0.1 fg/mL. In some embodiments, the reaction has a specificity of greater than about 75%.

In some embodiments, the method further comprises (c) ligating a first element of the first biomolecule and a second element of the second biomolecule using a ligase, thereby generating a ligated nucleic acid template across the first probe and the second probe. In some embodiments, the method further comprises (d) generating a complementary sequence of the ligated nucleic acid template.

In some embodiments, in (d), the generating the complementary sequence of the ligated nucleic acid template comprises thermocycling the ligated nucleic acid template. In some embodiments, in (d), the complementary sequence of the ligated nucleic acid template is positioned on the surface of the substrate. In some embodiments, in (d), the complementary sequence of the ligated nucleic acid template is released from the surface of the substrate. In some embodiments, the thermocycling comprises inactivating the ligase. In some embodiments, the thermocycling comprises using a polymerase.

In some embodiments, the method further comprises (e) amplifying the complementary sequence of the ligated nucleic acid template, thereby generating a nucleic acid molecule. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction. In some embodiments, the method further comprises (f) sequencing the nucleic acid molecule. In some embodiments, the substrate is fluorescently labeled. In some embodiments, the sequencing is conducted on the fluorescently labeled substrate. Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1, Panels A-D exemplify a sample workflow of proximity ligation assay (PLA).

FIG. 2 exemplifies the concept of ligation in PLA to produce a detectable signal when two or more probes come into close proximity.

FIG. 3, Panels A-E show an example of a PLA assay probe design.

FIG. 4 shows the PLA sources of intended signal and sources of background/noise and loss of signal due to unintended interactions.

FIG. 5 shows a schematic of PLA oligonucleotide components.

FIG. 6 shows a schematic of antibody conjugation and purification workflow.

FIG. 7, Panel A and B show the relationship between cycle thresholds and ligation events vs. number of beads.

FIG. 8, Panels A-F outline the general operations of a PLA assay and the molecular interactions occurring.

FIG. 9, Panels A and B show a digital PCR quantitation of a 7-point standard curve using commercially available reference material without background subtraction (FIG. 9, Panel A) and with background subtraction (FIG. 9, Panel B).

FIG. 10 shows the differentiation of small changes in a qPCR system vs. the platform digital PLA system.

FIG. 11 shows a computer system that is programmed or otherwise configured to implement methods provided herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive surface area.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It can be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and sub combinations of ranges and specific embodiments therein are intended to be included. The term “about” or “approximately” when referring to a number or a numerical range may refer that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

Recognized herein is a need for improved systems and compositions and methods to increase sensitivity of assays designed to detect and measure rare targets by controlling the ratio of components to specifically reduce background signal levels. The present disclosure provides systems and compositions and methods to implement using proximity ligation assay (PLA) and can be applied to any technique that utilizes the proximity of two or more probes, or combination of two or more detectors. Example readouts may include quantitative polymerase chain reaction (qPCR) and digital polymerase chain reaction (dPCR). In some aspects, the present disclosure describes one or more systems and methods for improved biomarker detection.

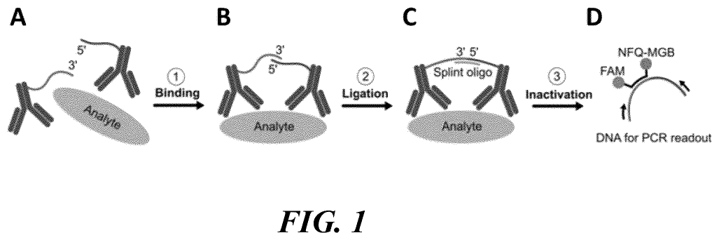

Proximity ligation assay (PLA) is an immunoassay technique that combines the specificity of antibody detection with PCR-based signal amplification. The process may begin by selecting a pair of probes that specifically target the protein of interest, as shown in FIG. 1, Panels A-D. A probe may be conjugated with a unique oligonucleotide sequence, shown as strands in FIG. 1, Panel A. When the two probes bind to the target protein in the sample, their attached DNA strands may be brought into close proximity, as shown in FIG. 1, Panel B. Upon adding the sample-probe mixture to a polymerase chain reaction (PCR) master mix containing ligase, the enzyme may ligate the two adjacent DNA strands, as shown in FIG. 1, Panel C. The ligation event, enabled by the colocalization of the probes, may create a single continuous DNA strand. The newly formed DNA strand may be amplified through downstream PCR processes, as shown in FIG. 1, Panel D.

Incorporation of PCR polymerase in PLA may enable exponential amplification of the target signal, enhancing sensitivity and allowing for the detection of low-abundance proteins that may be undetectable by standard methods. However, limitations still exist within the PLA framework. Quantification remains largely relative, necessitating standard materials for accurate readout. The amplification capacity of PCR polymerase may inadvertently amplify background noise, negatively impacting sensitivity if non-specific binding or contamination occurs. This risk underscores the importance of careful assay design and stringent control measures to mitigate the potential amplification of background signals, ensuring both high sensitivity and accuracy. Furthermore, the homogeneous implementation of PLA may exhibit variability in sensitivity, particularly with blood-based samples, where inhibitors and interfering substances may affect enzyme function.

Recognized herein is a need for improved systems, compositions, and methods to increase sensitivity of assays designed to detect and measure rare targets by controlling the probe-to-substrate surface area ratio. For example, by controlling the density of the probe on substrate surface to correspond to a certain percentage surface area occupancy to minimize background signal and enable reliable detection of critical biomarkers. By intentionally controlling the surface area occupancy (e.g., the portion of the substrate surface area that is occupied by the surface area of probes or target molecules that bind to the substrate) such that a first probe occupies a small percentage on the substrate surface, unintended colocalization events may be reduced. In instances where there are a few first probes on a substrate surface, when a second probe is added and adsorbs non-specifically onto the substrate surface, it is much more likely that the second probe lands far away from the first probes and unable to trigger a ligation reaction. As a result, these approaches selectively suppress background noise without compromising the signal from target proteins, making them particularly valuable for detecting low-abundance biomarkers in complex biological matrices, such as serum and plasma. In addition, fewer washes on the substrate surface may be performed, which can reduce sample volume and decrease the overall reaction time from sample loading to detection of target. The same substrate surface can be used to perform binding of the target to the probes (e.g., generation of the complex) or binding of a formed complex to the surface and then perform a reaction between probes. Furthermore, downstream digital output technologies, such as digital PCR, can be utilized to enhance precision in detecting subtle sample differences. This ultrasensitive approach has significant applications in diagnostics, enabling the reliable measurement of small differences, which is crucial for longitudinal monitoring of disease prognosis and staging.

In one aspect, the present disclosure provides a method for analyte detection, comprising (a) providing a complex coupled to a surface of a substrate. In some embodiments, the complex comprises (i) an analyte. In some embodiments, the complex comprises (ii) a first probe coupled to the analyte. In some embodiments, the complex comprises (iii) a second probe coupled to the analyte. In some embodiments, the first probe or the second probe is coupled to the surface of the substrate. In some embodiments, the method comprises (b) using the first probe and the second probe to generate a reaction product. In some embodiments, the method comprises (c) detecting the analyte using the reaction product. In some embodiments, the method comprises (c) detecting the analyte using the reaction product with a detection limit of about 0.1 fg/mL or less for detecting the analyte. In some embodiments, (a)-(c) are performed in no more than 90 minutes. The method can further comprise, prior to (b), washing the complex coupled to the surface of the substrate at most once.

In another aspect, the present disclosure provides a method for analyte detection, comprising (a) providing (i) a first probe coupled to a surface of a substrate. In some embodiments, (a) providing (ii) the analyte. In some embodiments, the method comprises (b) binding the first probe coupled to the surface of the substrate and a second probe to the analyte, to generate a complex. In some embodiments, the method comprises (c) using the first probe and the second probe of the complex to generate a reaction product. In some embodiments, the method comprises (d) detecting the analyte using the reaction product. In some embodiments, in (c), the complex is coupled to the surface of the substrate. In some embodiments, the method further comprises, in (d), detecting the analyte using the reaction product with a detection limit of about 0.1 fg/mL or less for detecting the analyte. In some embodiments, the method further comprises, prior to (c), washing the complex at most once.

In another aspect, the present disclosure provides a method for analyte detection, comprising (a) providing a complex coupled to a surface of a substrate. In some embodiments, the complex comprises (i) an analyte. In some embodiments, the complex comprises (ii) a first probe coupled to the analyte. In some embodiments, the complex comprises (iii) a second probe coupled to the analyte. In some embodiments, the first probe or the second probe is coupled to the surface of the substrate. In some embodiments, the method comprises (b) using the first probe and the second probe to generate a reaction product. In some embodiments, the method comprises, prior to (b), the complex coupled to the surface of the substrate is washed at most once. In some embodiments, the method further comprises, in (c), detecting the analyte using the reaction product with a detection limit of about 0.1 fg/mL or less for detecting the analyte. In some embodiments, in (b), the complex is coupled to the surface of the substrate.

In another aspect, the present disclosure provides a method for analyte detection, comprising (a) generating a complex. In some embodiments, the complex comprises (i) an analyte. In some embodiments, the complex comprises (ii) a first probe coupled to the analyte. In some embodiments, the complex comprises (iii) a second probe coupled to the analyte. In some embodiments, the method further comprises (b) without washing the complex after (a), coupling the complex generated in (a) to a surface of a substrate. In some embodiments, the method further comprises (c) using the first probe and the second probe of the complex coupled to the surface of the substrate to generate a reaction product. In some embodiments, the method further comprises (d) detecting the analyte using the reaction product. In some embodiments, the method further comprises, in (d), detecting the analyte using the reaction product with a detection limit of about 0.1 fg/mL or less for detecting the analyte. In some embodiments, the method further comprises, prior to (c), washing the complex coupled to the surface of the substrate at most once. In some embodiments, (i) the first probe or the second probe is coupled to the surface of the substrate. In some embodiments, (ii) a density of the first probe or the second probe coupled to the surface of the substrate corresponds to no more than about 5% surface area occupancy of the surface. In some embodiments, (i) the first probe or the second probe is coupled to the surface of the substrate. In some embodiments, (ii) the first probe or the second probe is coupled to the surface of the substrate via a binding pair.

In various aspects, (a)-(c) are performed in no more than about 90 minutes. In some embodiments, (a)-(c) are performed in no more than about 60 minutes. In some embodiments, (a)-(c) are performed in no more than about 30 minutes. In some embodiments, (a)-(d) are performed in no more than about 90 minutes. In some embodiments, (a)-(d) are performed in no more than about 60 minutes. In some embodiments, (a)-(d) are performed in no more than about 30 minutes. In some embodiments, in (a), a density of the first probe or the second probe coupled to the surface of the substrate corresponds to no more than about 5% surface area occupancy of the surface. In some embodiments, the first probe or the second probe is coupled to the surface of the substrate via a binding pair. In some embodiments, the first probe is coupled to the surface of the substrate via a binding pair. In some embodiments, the binding pair comprises biotin-streptavidin. In some embodiments, the surface is selected from the group consisting of a solid surface, a bead, a micropost, a microfluidic element, and a coated well. In some embodiments, the first probe or the second probe comprises an antibody. In some embodiments, the first probe or the second probe comprises an aptamer. In some embodiments, the first probe or the second probe comprises an antigen. In some embodiments, the analyte is a biomolecule. In some embodiments, the biomolecule is a protein. In some embodiments, in (b), the reaction product comprises a fluorescence resonance energy transfer (FRET) donor or acceptor. In some embodiments, in (c), the reaction product comprises a fluorescence resonance energy transfer (FRET) donor or acceptor. In some embodiments, the method further comprises detecting the analyte using the reaction product with a specificity of greater than about 75%. In some embodiments, (i) the first probe comprises a first binding moiety coupled to a first biomolecule; and (ii) the second probe comprises a second binding moiety coupled to a second biomolecule. In some embodiments, (b) comprises reacting the first biomolecule with the second biomolecule to couple the first biomolecule to the second biomolecule. In some embodiments, (c) comprises reacting the first biomolecule with the second biomolecule to couple the first biomolecule to the second biomolecule. In some embodiments, the first biomolecule is a first nucleic acid molecule and the second biomolecule is a second nucleic acid molecule. In some embodiments, (b) comprises ligating a first element of the first nucleic acid molecule to a second element of the second nucleic acid molecule thereby generating a ligated nucleic acid molecule, wherein the ligated nucleic acid molecule couples the first probe to the second probe. In some embodiments, (c) comprises ligating a first element of the first nucleic acid molecule to a second element of the second nucleic acid molecule thereby generating a ligated nucleic acid molecule, wherein the ligated nucleic acid molecule couples the first probe to the second probe. In some embodiments, the method further comprises generating a complementary nucleic acid molecule of the ligated nucleic acid molecule. In some embodiments, the method further comprises amplifying the complementary nucleic acid molecule of the ligated nucleic acid molecule, thereby generating the reaction product. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction. In some embodiments, (c) comprises sequencing the reaction product. In some embodiments, (d) comprises sequencing the reaction product. In some embodiments, the substrate is fluorescently labeled.

In another aspect, the present disclosure provides a composition, comprising a plurality of probes. In some embodiments, (a) a first probe of the plurality of probes comprises (1) a first binding moiety. In some embodiments, (a) the first probe of the plurality of probes comprises (2) a first biomolecule. In some embodiments, (a) a first probe of the plurality of probes comprises (1) a first binding moiety and (2) a first biomolecule. In some embodiments, (i) the first probe comprising the first biomolecule is coupled to a surface of a substrate, wherein a density of the first probe coupled to the surface of the substrate corresponds to no more than about 5% surface area occupancy of the surface. In some embodiments, (ii) the first binding moiety is configured to bind to an analyte. In some embodiments, (b) a second probe of the plurality of probes comprises (1) a second binding moiety. In some embodiments, (b) the second probe of the plurality of probes comprises (2) a second biomolecule. In some embodiments, (b) a second probe of the plurality of probes comprises (1) a second binding moiety and (2) a second biomolecule. In some embodiments, the second binding moiety is configured to bind to the analyte. In some embodiments, the first probe is coupled to the surface of the substrate via a binding pair.

In another aspect, the present disclosure provides a composition, comprising a plurality of probes. In some embodiments, (a) a first probe of the plurality of probes comprises (1) a first binding moiety covalently attached to a first member of a binding pair. In some embodiments, (a) a first probe of the plurality of probes comprises (2) a first nucleic acid molecule. In some embodiments, (a) a first probe of the plurality of probes comprises (1) a first binding moiety covalently attached to a first member of a binding pair and (2) a first nucleic acid molecule. In some embodiments, the first binding moiety is configured to bind to an analyte. In some embodiments, the composition further comprises (b) a surface of a substrate comprising a second member of the binding pair. In some embodiments, the method further comprises (c) a second probe of the plurality of probes comprises (1) a second binding moiety. In some embodiments, the method further comprises (c) a second probe of the plurality of probes comprises (2) a second nucleic acid molecule. In some embodiments, the method further comprises (c) a second probe of the plurality of probes comprises (1) a second binding moiety and (2) a second nucleic acid molecule. In some embodiments, the second binding moiety is configured to bind to the analyte. In some embodiments, the first member of the binding pair is bound to the second member of the binding pair, such that the first probe is bound to the surface of the substrate. In some embodiments, a density of the first probe bound on the surface of the substrate corresponds to no more than about 5% surface area occupancy of the surface.

In various aspect, the binding pair comprises biotin-streptavidin. In some embodiments, the surface is selected from the group consisting of a solid surface, a bead, a micropost, a microfluidic element, and a coated well. In some embodiments, the first binding moiety or the second binding moiety comprises an antibody. In some embodiments, the first binding moiety or the second binding moiety comprises an aptamer. In some embodiments, the first binding moiety or the second binding moiety comprises an antigen. In some embodiments, the analyte is a biomolecule. In some embodiments, the biomolecule is a protein. In some embodiments, the composition further comprises, the analyte, wherein the first binding moiety and the second binding moiety are bound to the analyte. In some embodiments, the first biomolecule and the second biomolecule are positioned within sufficient distance to participate in a reaction with one another. In some embodiments, the first biomolecule and the second biomolecule are nucleic acid molecules. In some embodiments, the first binding moiety and the second binding moiety are antibodies. In some embodiments, the first biomolecule is a fluorescence resonance energy transfer (FRET) donor or acceptor. In some embodiments, the method further comprises a ligase. In some embodiments, the substrate is fluorescently labeled.

In another aspect, the present disclosure provides a method of detecting an analyte, comprising (a) providing a complex coupled to a surface of a substrate. In some embodiments, the complex comprises (A) a first probe comprising (1) a first binding moiety. In some embodiments, the complex comprises (A) a first probe comprising (2) a first biomolecule. In some embodiments, the complex comprises (A) a first probe comprising (1) a first binding moiety and (2) a first biomolecule. In some embodiments, (i) the first probe comprises the first biomolecule and is coupled to the surface of the substrate. In some embodiments, (ii) a density of the first probe coupled to the surface of the substrate corresponds to no more than about 5% surface area occupancy of the surface. In some embodiments, (iii) the first binding moiety is bound to an analyte. In some embodiments, the complex comprises (B) a second probe comprising (1) a second binding moiety. In some embodiments, the complex comprises (B) a second probe comprising (2) a second biomolecule. In some embodiments, the complex comprises (B) a second probe comprising (1) a second binding moiety and (2) a second biomolecule. In some embodiments, the second binding moiety is bound to the analyte. In some embodiments, the method further comprises (b) using the first probe and the second probe to generate a reaction product. In some embodiments, the method further comprises (c) detecting the analyte using the reaction product. In some embodiments, the first probe is coupled to the surface of the substrate via a binding pair. In some embodiments, (b) comprises reacting the first biomolecule with the second biomolecule to couple the first biomolecule to the second biomolecule. In some embodiments, the first biomolecule is a first nucleic acid molecule and the second biomolecule is a second nucleic acid molecule.

In another aspect, the present disclosure provides a method of detecting an analyte, comprising (a) providing a complex on a surface of a substrate. In some embodiments, the complex comprises (A) a first probe comprising (1) a first binding moiety covalently attached to a first member of a binding pair. In some embodiments, the complex comprises (A) a first probe comprising (2) a first nucleic acid molecule. In some embodiments, the complex comprises (A) a first probe comprising (1) a first binding moiety covalently attached to a first member of a binding pair and (2) a first nucleic acid molecule. In some embodiments, the first binding moiety is bound to an analyte. In some embodiments, the complex comprises (B) the surface of the substrate comprising a second member of the binding pair, the second member of the binding pair bound to the first member of the binding pair such that the first probe is bound to the surface. In some embodiments, the complex comprises (C) a second probe comprising (1) a second binding moiety. In some embodiments, the complex comprises (C) a second probe comprising (2) a second nucleic acid molecule. embodiments, the complex comprises (C) a second probe comprising (1) a second binding moiety and (2) a second nucleic acid molecule. In some embodiments, the second binding moiety is configured to bind to the analyte. In some embodiments, the method further comprises (b) using the first probe and the second probe to generate a reaction product. In some embodiments, the method further comprises (c) detecting the analyte using the reaction product. In some embodiments, a density of the first probe bound to the surface of the substrate corresponds to no more than about 5% surface area occupancy of the surface. In some embodiments, (b) comprises reacting the first nucleic acid molecule with the second nucleic acid molecule to couple the first nucleic acid molecule to the second nucleic acid molecule.

In various aspects, in (c), detecting the analyte using the reaction product with a detection limit of about 0.1 fg/mL or less for detecting the analyte. In some embodiments, in (c), detecting the analyte using the reaction product with a specificity of greater than about 75%. In some embodiments, the method further comprises, prior to (b), washing the complex coupled to the surface of the substrate at most once. In some embodiments, (a)-(c) are performed in no more than about 90 minutes. In some embodiments, (a)-(c) are performed in no more than about 60 minutes. In some embodiments, (a)-(c) are performed in no more than about 30 minutes. In some embodiments, the binding pair comprises biotin-streptavidin. In some embodiments, the surface is selected from the group consisting of a solid surface, a bead, a micropost, a microfluidic element, and a coated well. In some embodiments, the first probe or the second probe comprises an antibody. In some embodiments, the first probe or the second probe comprises an aptamer. In some embodiments, the first probe or the second probe comprises an antigen. In some embodiments, the analyte is a biomolecule. In some embodiments, the biomolecule is a protein. In some embodiments, in (b), the reaction product comprises a fluorescence resonance energy transfer (FRET) donor or acceptor. In some embodiments, (b) comprises ligating a first element of the first nucleic acid molecule to a second element of the second nucleic acid molecule thereby generating a ligated nucleic acid molecule, wherein the ligated nucleic acid molecule couples the first probe to the second probe. In some embodiments, the method further comprises generating a complementary nucleic acid molecule of the ligated nucleic acid molecule. In some embodiments, the method further comprises amplifying the complementary nucleic acid molecule of the ligated nucleic acid molecule, thereby generating the reaction product. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction. In some embodiments, (c) comprises sequencing the reaction product. In some embodiments, the substrate is fluorescently labeled.

In various aspects, the first biomolecule identifies the first binding moiety. In some embodiments, the second biomolecule identifies the second binding moiety.

In various aspects, the first probe or the second probe is coupled to the surface of the substrate via a binding pair. In some embodiments, the first member of the binding pair comprises biotin. In some embodiments, the second member of the binding pair comprises streptavidin. In some embodiments, the surface is selected from the group consisting of a solid surface, a bead, a micropost, a microfluidic element, and a coated well. In some embodiments, the surface is a solid surface. In some embodiments, the surface comprises a bead. In some embodiments, the surface comprises a micropost. In some embodiments, the surface comprises a microfluidic element. In some embodiments, the surface comprises a coated well. In some embodiments, the first probe or the second probe comprises an antibody. In some embodiments, the first binding moiety or the second binding moiety comprises an antibody. In some embodiments, the first probe or the second probe comprises an aptamer. In some embodiments, wherein the first binding moiety or the second binding moiety comprises an aptamer. In some embodiments, the first probe or the second probe comprises an antigen. In some embodiments, the first binding moiety or the second binding moiety comprises an antigen. In some embodiments, the first probe and the second probe comprise antibodies. In some embodiments, the first binding moiety and the second binding moiety comprise antibodies. In some embodiments, the first probe and the second probe comprise aptamers. In some embodiments, wherein the first binding moiety and the second binding moiety comprise aptamers. In some embodiments, the first probe and the second probe comprise antigens. In some embodiments, the first binding moiety and the second binding moiety comprise antigens. In some embodiments, the first binding moiety is configured to bind to an epitope of the analyte. In some embodiments, the second binding moiety is configured to bind to a different epitope of the analyte.

In some embodiments, the method comprises using the first probe and the second probe to generate a reaction product. In some embodiments, the reaction product comprises a fluorescence resonance energy transfer (FRET) donor or acceptor.

In some embodiments, using the first probe and the second probe to generate a reaction product comprises performing an assay. In some embodiments, the assay comprises a proximity ligation assay (PLA). In some embodiments, the assay comprises a colocalization reaction. In some embodiments, the assay comprises a fluorescence resonance energy transfer (FRET) assay. In some embodiments, the reaction product can be used as a template for polymerase chain reaction. In some embodiments, the reaction product can comprise a double stranded DNA (dsDNA).

In some embodiments, the first biomolecule and the second biomolecule are positioned within sufficient distance to participate in a reaction with one another. In some embodiments, the first nucleic acid molecule and the second nucleic acid molecule are positioned within sufficient distance to participate in a reaction with one another. In some embodiments, the reaction comprises performing a proximity ligation assay (PLA). In some embodiments, the reaction comprises performing a colocalization reaction. In some embodiments, the reaction comprises performing a fluorescence resonance energy transfer (FRET) assay.

In some embodiments, the method comprises detecting the analyte using the reaction product. In some embodiments, detecting the analyte using the reaction product with a detection limit of about 0.1 fg/mL or less for detecting the analyte. In some embodiments, detecting the analyte using the reaction product with a specificity of greater than about 75%.

In some embodiments, the first probe comprises a first binding moiety coupled to a first biomolecule. In some embodiments, the second probe comprises a second binding moiety coupled to a second biomolecule. In some embodiments, the method further comprises reacting the first biomolecule with the second biomolecule to couple the first biomolecule to the second biomolecule. In some embodiments, the first biomolecule is a first nucleic acid molecule and the second biomolecule is a second nucleic acid molecule. In some embodiments, the method further comprises ligating a first element of the first nucleic acid molecule to a second element of the second nucleic acid molecule thereby generating a ligated nucleic acid molecule, wherein the ligated nucleic acid molecule couples the first probe to the second probe.

In some embodiments, the method further comprises generating a complementary nucleic acid molecule of the ligated nucleic acid molecule. In some embodiments, the generating the complementary nucleic acid molecule of the ligated nucleic acid molecule comprises thermocycling the ligated nucleic acid molecule. In some embodiments, the complementary nucleic acid molecule of the ligated nucleic acid molecule is positioned on the surface of the substrate. In some embodiments, complementary nucleic acid molecule of the ligated nucleic acid molecule is released from the surface of the substrate. In some embodiments, the thermocycling comprises inactivating the ligase. In some embodiments, the thermocycling comprises using a polymerase.

In various aspects, the method can further comprise amplifying complementary nucleic acid molecule of the ligated nucleic acid molecule, thereby generating a reaction product. In some embodiments, the amplifying comprises performing a polymerase chain reaction. In some embodiments, the polymerase chain reaction is a digital polymerase chain reaction. In some embodiments, the polymerase chain reaction is a quantitative polymerase chain reaction.

In various aspects, the method can further comprise sequencing the reaction product. In some embodiments, the substrate is fluorescently labeled.

In some embodiments, a first binding moiety can be an antibody. In some embodiments, the first binding moiety can be an aptamer. In some embodiments, the first binding moiety can be an antigen. In some embodiments, the first binding moiety can be a nucleic acid. In some embodiments, the nucleic acid may be, for example, deoxyribonucleic acid (DNA), ribonucleic acid (RNA), or DNA copies of ribonucleic acid (cDNA). The RNA may be, for example, messenger RNA (mRNA), transfer RNA (tRNA), ribosomal RNA (rRNA), guide RNAs (gRNAs), small nuclear RNA (snRNA), small nucleolar RNA (snoRNA), SmY RNA, spliced leader RNA (SL RNA), CRISPR RNA, long noncoding RNA (lncRNA), microRNA (miRNA), or another suitable RNA. In some embodiments, the nucleic acid may be single stranded. In some embodiments, the nucleic acid may be double stranded.

In some embodiments, a second binding moiety is an antibody. In some embodiments, the second binding moiety is an aptamer. In some embodiments, the second binding moiety is an antigen. In some embodiments, the second binding moiety is a nucleic acid. In some embodiments, the nucleic acid may be, for example, deoxyribonucleic acid (DNA), ribonucleic acid (RNA), or DNA copies of ribonucleic acid (cDNA). The RNA may be, for example, messenger RNA (mRNA), transfer RNA (tRNA), ribosomal RNA (rRNA), guide RNAs (gRNAs), small nuclear RNA (snRNA), small nucleolar RNA (snoRNA), SmY RNA, spliced leader RNA (SL RNA), CRISPR RNA, long noncoding RNA (lncRNA), microRNA (miRNA), or another suitable RNA. In some embodiments, the nucleic acid may be single stranded. In some embodiments, the nucleic acid may be double stranded.

In some embodiments, a composition may comprise a plurality of binding moieties. In some embodiments, a plurality of binding moieties may comprise any number of binding moieties, for example, a first binding moiety, a second binding moiety, a third binding moiety, a fourth binding moiety, a fifth binding moiety, a sixth binding moiety, a seventh binding moiety, an eight binding moiety, a ninth binding moiety, a tenth binding moiety. A binding moiety may comprise different positions for different molecular components to couple to the binding moiety. For example, a biomolecule and a member of a binding pair may be coupled to a first binding moiety at different positions of the first binding moiety. In some embodiments, a binding moiety may be coupled to a biomolecule. In some embodiments, a binding moiety may be coupled to a member of an analyte. In some embodiments, a binding moiety may be configured to bind to a member of an analyte.

In some embodiments, a first biomolecule is an oligonucleotide. In some embodiments, the first biomolecule is a nucleic acid. In some embodiments, the nucleic acid may be, for example, deoxyribonucleic acid (DNA), ribonucleic acid (RNA), or DNA copies of ribonucleic acid (cDNA). The RNA may be, for example, messenger RNA (mRNA), transfer RNA (tRNA), ribosomal RNA (rRNA), guide RNAs (gRNAs), small nuclear RNA (snRNA), small nucleolar RNA (snoRNA), SmY RNA, spliced leader RNA (SL RNA), CRISPR RNA, long noncoding RNA (lncRNA), microRNA (miRNA), or another suitable RNA. In some embodiments, the nucleic acid may be single stranded. In some embodiments, the nucleic acid may be double stranded. In some embodiments, the first biomolecule is a light-sensitive molecule. In some embodiments, the first biomolecule is a chromophore.

In some embodiments, a second biomolecule is an oligonucleotide. In some embodiments, the second biomolecule is a nucleic acid. In some embodiments, the nucleic acid may be, for example, deoxyribonucleic acid (DNA), ribonucleic acid (RNA), or DNA copies of ribonucleic acid (cDNA). The RNA may be, for example, messenger RNA (mRNA), transfer RNA (tRNA), ribosomal RNA (rRNA), guide RNAs (gRNAs), small nuclear RNA (snRNA), small nucleolar RNA (snoRNA), SmY RNA, spliced leader RNA (SL RNA), CRISPR RNA, long noncoding RNA (lncRNA), microRNA (miRNA), or another suitable RNA. In some embodiments, the nucleic acid may be single stranded. In some embodiments, the nucleic acid may be double stranded. In some embodiments, the second biomolecule is a light-sensitive molecule. In some embodiments, the second biomolecule is a chromophore. In some embodiments, a biomolecule may be coupled to a binding moiety.

In some embodiments, a probe comprises, for example, a nucleic acid probe, a protein probe, an antibody probe, or a small molecule probe. The probe can comprise DNA, RNA, protein, or a modified nucleotide. In some embodiments, a probe may be a molecule designed to detect a specific component within a sample. In some embodiments, a probe may be a single-stranded sequence of DNA or RNA that is used to identify specific sequences of DNA or RNA. In some embodiments, a probe comprises an antibody. In some embodiments, a probe comprises an aptamer. In some embodiments, a probe comprises an antigen. In some embodiments, a probe may be detectable via chemical tags. In some embodiments, a chemical tag may be, for example, a radioactive isotope, enzyme, or fluorescent dye. In some embodiments, a plurality of probes may comprise more than one probe. In some embodiments, a plurality of probes may comprise a first probe of the plurality of probes. In some embodiments, a plurality of probes may comprise a second probe of the plurality of probes. In some embodiments, a plurality of probes may comprise a first probe of the plurality of probes and a second probe of the plurality of probes. In some embodiments, a probe comprising a binding moiety may be coupled to a biomolecule. In some embodiments, a probe comprising a biomolecule may be coupled to a binding moiety.

In some embodiments, a composition may comprise a plurality of probes. In some embodiments, a plurality of probes may comprise more than one probe. In some embodiments, a plurality of probes may comprise a first probe of the plurality of probes. In some embodiments, a plurality of probes may comprise a second probe of the plurality of probes. In some embodiments, a plurality of probes may comprise a third probe of the plurality of probes. In some embodiments, a plurality of probes may comprise a first probe of the plurality of probes and a second probe of the plurality of probes. In some embodiments, a plurality of probes may comprise a first probe of the plurality of probes, a second probe of the plurality of probes, and a third probe of the plurality of probes.

In some embodiments, the third probe of the plurality of probes may be configured to bind to the second probe of the plurality of probes. In some embodiments, the first probe of the plurality of probes does not contain a biomolecule. In some embodiments, the second probe of the plurality of probes does not contain a biomolecule. In some embodiments, the third probe of the plurality of probes does not contain a biomolecule. In some embodiments, the first probe of the plurality of probes comprises a plurality of biomolecules. In some embodiments, the second probe of the plurality of probes comprises a plurality of biomolecules. In some embodiments, the third probe of the plurality of probes comprises a plurality of biomolecules. In some embodiments, the first probe of the plurality of probes comprises a plurality of binding moieties. In some embodiments, the second probe of the plurality of probes comprises a plurality of binding moieties. In some embodiments, the third probe of the plurality of probes comprises a plurality of binding moieties. In some embodiments, the second probe of the plurality of probes and the third probe of the plurality of probes is at a ratio of greater than, for example, 0.01, 0.1, 0.2, 0.3, 0.4, 0.5, 1, 5, 10, 20, 25, or 50. In some embodiments, the second probe of the plurality of probes and the third probe of the plurality of probes is at a ratio of greater than, for example, 1%, 10%, 20%, 30%, 40%, 50%, 100%, 500%, 10000%, 20000%, 25000%, or 50000%. In some embodiments, the second probe of the plurality of probes and the third probe of the plurality of probes is at a ratio of less than, for example, 0.01, 0.1, 0.2, 0.3, 0.4, 0.5, 1, 5, 10, 20, 25, 50, 75, 80, 85, 90, 95, 99, or 100. In some embodiments, the second probe of the plurality of probes and the third probe of the plurality of probes is at a ratio of less than, for example, 1%, 10%, 20%, 30%, 40%, 50%, 100%, 500%, 10000%, 20000%, 25000%, 50000%, 75000%, 80000%, 85000%, 90000%, 95000%, 99000%, or 100000%. In some embodiments, the first probe of the plurality of probes and the second probe of the plurality of probes is at a ratio of greater than, for example, 0.01, 0.1, 0.2, 0.3, 0.4, 0.5, 1, 5, 10, 20, 25, or 50. In some embodiments, the first probe of the plurality of probes and the second probe of the plurality of probes is at a ratio of greater than, for example, 1%, 10%, 20%, 30%, 40%, 50%, 100%, 500%, 10000%, 20000%, 25000%, or 50000%. In some embodiments, the first probe of the plurality of probes and the second probe of the plurality of probes is at a ratio of less than, for example, 0.01, 0.1, 0.2, 0.3, 0.4, 0.5, 1, 5, 10, 20, 25, 50, 75, 80, 85, 90, 95, 99, or 100. In some embodiments, the first probe of the plurality of probes and the second probe of the plurality of probes is at a ratio of less than, for example, 1%, 10%, 20%, 30%, 40%, 50%, 100%, 500%, 10000%, 20000%, 25000%, 50000%, 75000%, 80000%, 85000%, 90000%, 95000%, 99000%, or 100000%. In some embodiments, the first probe of the plurality of probes and the third probe of the plurality of probes is at a ratio of greater than, for example, 0.01, 0.1, 0.2, 0.3, 0.4, 0.5, 1, 5, 10, 20, 25, or 50. In some embodiments, the first probe of the plurality of probes and the third probe of the plurality of probes is at a ratio of greater than, for example, 1%, 10%, 20%, 30%, 40%, 50%, 100%, 500%, 10000%, 20000%, 25000%, or 50000%. In some embodiments, the first probe of the plurality of probes and the third probe of the plurality of probes is at a ratio of less than, for example, 0.01, 0.1, 0.2, 0.3, 0.4, 0.5, 1, 5, 10, 20, 25, 50, 75, 80, 85, 90, 95, 99, or 100. In some embodiments, the first probe of the plurality of probes and the third probe of the plurality of probes is at a ratio of less than, for example, 1%, 10%, 20%, 30%, 40%, 50%, 100%, 500%, 10000%, 20000%, 25000%, 50000%, 75000%, 80000%, 85000%, 90000%, 95000%, 99000%, or 100000%.

In some embodiments, a binding pair may be a pair of molecules that have strong association with each other. In some embodiments, a binding pair may comprise a first member of the binding pair. In some embodiments, a binding pair may comprise a second member of the binding pair. In some embodiments, the binding pair may have a strong binding affinity with each other. In some embodiments, the binding pair comprises avidin and biotin. In some embodiments, the binding pair comprises streptavidin and biotin. In some embodiments, the binding pair comprises antibody and antigen. In some embodiments, the binding pair may comprise a first member. In some embodiments, the binding pair may comprise a second member. In some embodiments, the first member of the binding pair comprises biotin. In some embodiments, the second member of the binding pair comprises streptavidin.

In some embodiments, a substrate may comprise a surface on which a molecule binds to. In some embodiments, the surface may be a solid surface. In some embodiments, the surface may comprise, for example, a bead, a micropost, a microfluidic element, or a coated well. In some embodiments, the microfluidic element may comprise a channel, or a chamber.

In some embodiments, an analyte can be a substance, component, compound, or molecule of interest that is being measured, detected, analyzed in a scientific experiment or analytical test. In some embodiments, the analyte can be a molecule (e.g., a protein, a nucleic acid molecule, a metabolite, an ion, a biomolecule, a peptide, an antibody, a biomarker, a carbohydrate, a small molecule) that can be present in a sample and can be a target of measurement or detection. In some embodiments, the analyte is a biomolecule. In some embodiments, the biomolecule is a protein. In some embodiments, the analyte can be an antibody. In some embodiments, the analyte can be a biomarker. In some embodiments, the analyte can be, for example, p-Tau 217, a phosphorylated variant, p-Tau 181, an unphosphorylated Tau isoform, Aβ42, an amyloid peptide, Aβ38, or Aβ40.

In some embodiments, the analyte may have a plurality of epitopes. In some embodiments, the first binding moiety is configured to bind to an epitope of the analyte. In some embodiments, the second binding moiety is configured to bind to a different epitope of the analyte.

In some embodiments, a density may refer to the number of molecules or particles present in a given volume or area (e.g., a surface). For example, in a surface-based assay, density can describe how many probe or target molecules (e.g., binding moieties, antibodies, oligonucleotides) are attached to a unit area of the surface. For example, high density may refer to many molecules are bound to the surface, while low density may refer to fewer molecules are bound.

In some embodiments, a surface area occupancy may refer to the proportion of the substrate surface area is occupied by the surface area of probes or target molecules (e.g., binding moieties, antibodies, oligonucleotides) that bind to the substrate. The surface area occupancy can be important in assays, such as immunoassays or proximity ligation assays (PLAs), where the substrate surface can be functionalized with capture probes (e.g., antibodies, oligonucleotides) that interact with target molecules in the sample. In some embodiments, a surface area occupancy may refer to a fraction or percentage of the substate surface that is occupied, covered, or bound by probes or target molecules, as a result of, for example, adsorption, binding, or other molecular interactions. For example, a surface area occupancy (%) may be calculated multiplying the surface area covered by each probe by the number of probes, then dividing by the substrate surface area, e.g., [Surface Area of Each Probe x Number of Probes]/[Substrate Surface Area]. The surface area of each probe can be influenced by the size of each probe, and properties of the probe itself (e.g., size, geometry, shape, surface texture, surface modification, etc.). For example, if the probe is a sphere, the diameter is then used to project a circular area onto the surface of the substrate. The circular area projected may be the surface rea of the probe. In some embodiments, the total probe surface area may be calculated by multiplying the surface area of each probe by the number of probes. In some embodiments, the substrate surface area may be determined by the properties on the substrate itself (e.g., size, geometry, shape, surface texture, surface modification, bead size, microfluidic channel dimensions, etc.). In some embodiments, a low % surface area occupancy minimizes the chance a second probe is within sufficient distance to a first probe to participate in a reaction (e.g., proximity ligation assay) with one another. For example, minimizing the chance for a reaction to occur minimizes the generation of nonspecific background signal, as disclosed elsewhere herein.

In some embodiments, a density of a first probe on a surface of a substrate is no more than about 1% surface area occupancy, no more than about 2% surface area occupancy, no more than about 3% surface area occupancy, no more than about 4% surface area occupancy, no more than about 5% surface area occupancy, no more than about 6% surface area occupancy, no more than about 7% surface area occupancy, no more than about 8% surface area occupancy, no more than about 9% surface area occupancy, no more than about 10% surface area occupancy, no more than about 15% surface area occupancy, no more than about 20% surface area occupancy, no more than about 25% surface area occupancy, no more than about 30% surface area occupancy, no more than about 35% surface area occupancy, no more than about 40% surface area occupancy, no more than about 45% surface area occupancy, no more than about 50% surface area occupancy, no more than about 55% surface area occupancy, no more than about 60% surface area occupancy, no more than about 65% surface area occupancy, no more than about 70% surface area occupancy, no more than about 75% surface area occupancy, no more than about 80% surface area occupancy, no more than about 90% surface area occupancy, or no more than about 95% surface area occupancy of the surface. In some embodiments, a density of a first probe on a surface of a substrate is no less than about 0.01% surface area occupancy, no less than about 0.05% surface area occupancy, no less than about 0.1% surface area occupancy, no less than about 0.2% surface area occupancy, no less than about 0.3% surface area occupancy, no less than about 0.4% surface area occupancy, no less than about 0.5% surface area occupancy, no less than about 0.6% surface area occupancy, no less than about 0.7% surface area occupancy, no less than about 0.8% surface area occupancy, no less than about 0.9% surface area occupancy, no less than about 1% surface area occupancy, no less than about 2% surface area occupancy, no less than about 3% surface area occupancy, no less than about 4% surface area occupancy, no less than about 5% surface area occupancy, no less than about 6% surface area occupancy, no less than about 7% surface area occupancy, no less than about 8% surface area occupancy, no less than about 9% surface area occupancy, no less than about 10% surface area occupancy, no less than about 15% surface area occupancy, no less than about 20% surface area occupancy, no less than about 25% surface area occupancy, no less than about 30% surface area occupancy, no less than about 35% surface area occupancy, no less than about 40% surface area occupancy, no less than about 45% surface area occupancy, no less than about 50% surface area occupancy, no less than about 55% surface area occupancy, no less than about 60% surface area occupancy, no less than about 65% surface area occupancy, no less than about 70% surface area occupancy, no less than about 75% surface area occupancy, no less than about 80% surface area occupancy, no less than about 90% surface area occupancy, or no less than about 95% surface area occupancy of the surface.

In some embodiments, the first nucleic acid molecule and the second nucleic acid molecule are positioned within sufficient distance to participate in a reaction with one another. In some embodiments, the first biomolecule and the second biomolecule are positioned within sufficient distance to participate in a reaction with one another. In some embodiments, the sufficient distance may be about 1 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 150 nm, about 175 nm, or about 200 nm. In some embodiments, the sufficient distance may be no more than about 1 nm, no more than about 5 nm, no more than about 10 nm, no more than about 15 nm, no more than about 20 nm, no more than about 25 nm, no more than about 30 nm, no more than about 35 nm, no more than about 40 nm, no more than about 45 nm, no more than about 50 nm, no more than about 55 nm, no more than about 60 nm, no more than about 65 nm, no more than about 70 nm, no more than about 75 nm, no more than about 80 nm, no more than about 85 nm, no more than about 90 nm, no more than about 95 nm, no more than about 100 nm, no more than about 105 nm, no more than about 110 nm, no more than about 115 nm, no more than about 120 nm, no more than about 150 nm, no more than about 175 nm, or no more than about 200 nm. In some embodiments, the sufficient distance may be no less than about 1 nm, no less than about 5 nm, no less than about 10 nm, no less than about 15 nm, no less than about 20 nm, no less than about 25 nm, no less than about 30 nm, no less than about 35 nm, no less than about 40 nm, no less than about 45 nm, no less than about 50 nm, no less than about 55 nm, no less than about 60 nm, no less than about 65 nm, no less than about 70 nm, no less than about 75 nm, no less than about 80 nm, no less than about 85 nm, no less than about 90 nm, no less than about 95 nm, no less than about 100 nm, no less than about 105 nm, no less than about 110 nm, no less than about 115 nm, no less than about 120 nm, no less than about 150 nm, no less than about 175 nm, or no less than about 200 nm.

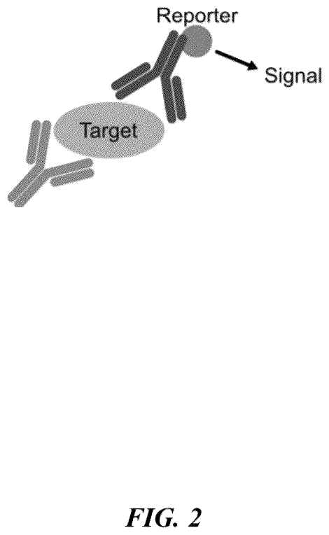

Without being bound by theory and as understood by a person skilled in the art, a proximity ligation assay (PLA) is an immunoassay method that integrates the specificity of antibody detection with PCR-based signal amplification. PLA can allow for the detection and quantification of specific biomolecular interactions, such as protein-protein, protein-DNA, or nucleic acid interactions, with high sensitivity and spatial resolution. PLA may utilize the concept of ligation to produce a detectable signal when two or more probes come into close proximity (e.g., 1-100 nm range), as shown in FIG. 2, facilitating specific and accurate detection of the target interaction. In some cases, PLA is based on the principle that when two specifically designed oligonucleotide-labeled probes bind to the target protein, their attached DNA strands can be brought into close proximity. In some cases, the two probes each comprise a binding moiety (e.g., an antibody, a nucleic acid probe, an aptamer, an antigen) that specifically binds to the target molecules of interest. Each probe may carry a unique oligonucleotide sequence attached at the 3′ or 5′ end. The proximity of the two probes, achieved through their specific interaction with the target, triggers a ligation event. A DNA ligase can then catalyzes the ligation of these probes to form a DNA template for further amplification. This ligation event, when successful, generates a reaction product (e.g., double stranded DNA) which can be detected by various signal amplification methods, such as rolling circle amplification (RCA), dPCR, or qPCR, enabling the sensitive identification of molecular interactions. The amplified signal can be detected using various methods, such as sequencing, fluorescence, chemiluminescence, or other optical detection systems. The resulting signal can be proportional to the amount of target interaction, which allows for quantification of the interaction in the sample.

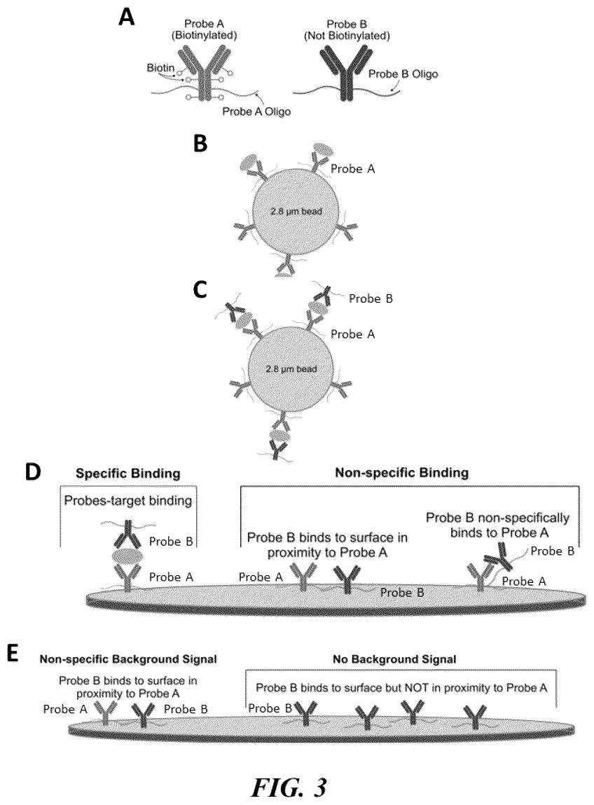

In one embodiment, a substrate may be used to specifically capture one of the antibodies (e.g., Probe A—comprising biotin, and Probe A oligonucleotide DNA)—e.g., streptavidin coated magnetic sphere+biotin—conjugated antibody, while a second antibody may be conjugated to a DNA strand (e.g., Probe B—comprising a different Probe B oligonucleotide DNA), as shown in FIG. 3, Panel A. Probe A may be incubated with the target-containing sample and the capture bead. After washing, Probe A may remain on the bead—some Probe A may have target bound while others will not, as shown in FIG. 3, Panel B. Subsequently, Probe B may be incubated with the above with the goal of binding Probe B to the target, as shown in FIG. 3, Panel C. In some embodiments, there may be two species bound to the bead-Probe A-Target-Probe B and Probe A alone. In some embodiments, a variety of other species may bind to the surface non-specifically. In some embodiments, Probe B may non-specifically adsorb to the bead and land close to Probe A, as shown in FIG. 3, Panel D. The resulting proximity may be sufficient to trigger the ligase and subsequent PCR to produce background signal. In some embodiments, the two probes may be DNA-conjugated antibodies. In some embodiments, the two probes may be other types of probes that may require proximity to produce signal.

In one aspect, a density or a ratio of Probe A on substrate or bead may be selected so that Probe As may occupy a small percentage of the substrate or bead surface (e.g., no more than about 5% surface area occupancy) in order to reduce background caused by unintended colocalization. In some cases, the surface area occupancy can be the portion of the substrate surface area that is occupied by the surface area of probes or target molecules that bind to the substrate, as described elsewhere herein. In some cases, when Probe B adsorbs non-specifically onto the bead and given that Probe As occupy a small percentage on the bead surface, it may be more likely that it lands faraway from a Probe A and may be unable to trigger the ligase, as shown in FIG. 3, Panel E. This may reduce the amount of background signal produced by reducing the likelihood the two probes will co-localize unintentionally. This effect may be called “spatial dilution,” by effectively achieving the functional dilution effect by spacing out the probes on a surface.

In addition, advantages of the compositions and methods for analyte detection described herein can include reduced washes on the complex coupled to the surface of the substrate, decreased reaction time from complex formation to analyte detection, and use of the same substrate surface to perform binding of the target to the probes (e.g., generation of the complex) or binding of a formed complex to the surface and then perform a reaction between probes. By intentionally controlling the density of a probe (e.g., Probe A) coupled to the surface of the substrate to minimize unintentional colocalization, the complex coupled to the surface of the substrate can be washed at most once prior to using the probes to generate a reaction product for downstream applications (e.g., dPCR, qPCR). With decreased washes, the overall reaction time from complex formation to analyte detection can be decreased (e.g., no more than 90 minutes).

In one aspect, a method for analyte detection comprises (a) providing a complex coupled to a surface of a substrate, wherein the complex comprises: (i) an analyte; (ii) a first probe coupled to the analyte; (iii) a second probe coupled to the analyte, wherein the first probe or the second probe is coupled to the surface of the substrate; (b) using the first probe and the second probe to generate a reaction product; and (c) detecting the analyte using the reaction product, with a detection limit of about 0.1 fg/mL or less for detecting the analyte. In some embodiments, (a)-(c) are performed in no more than about 90 minutes. In some embodiments, (a)-(c) are performed in no more than about 480 minutes, no more than about 420 minutes, no more than about 360 minutes, no more than about 300 minutes, no more than about 240 minutes, no more than about 180 minutes, no more than about 150 minutes, no more than about 120 minutes, no more than about 60 minutes, no more than about 45 minutes, no more than about 30 minutes, no more than about 15 minutes, no more than about minutes, no more than about 10 minutes, no more than about 5 minutes, or no more than about 1 minute. In some embodiments, the method comprises, prior to (b), the complex coupled to the surface of the substrate is washed at most once. In some embodiments, prior to (b), the complex coupled to the surface of the substrate is washed at most twice, at most three times, at most four times, at most five times, at most six times, at most seven times, at most eight times, at most nine times, at most ten times, or at most twenty times.

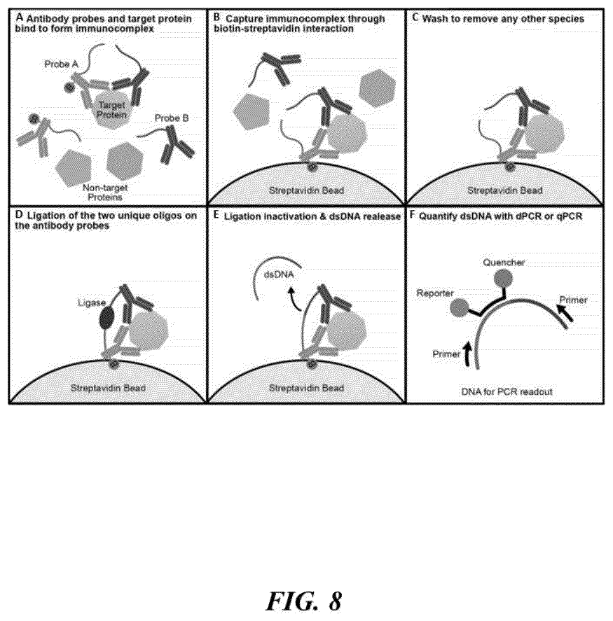

The method above describes a highly sensitive and fast analyte detection method that is agnostic to how the probe-analyte complex is formed. For example, as shown in FIG. 3, Panel C and FIG. 8, Panel A and described elsewhere herein, a complex comprising an analyte with a first probe coupled to the analyte and a second probe coupled to the analyte. The complex can be formed in solution (as shown in FIG. 8, Panel A) or on the surface of the substrate (as shown in FIG. 3, Panel C). After the first probe or the second probe is coupled to the surface of the substrate (as shown in FIG. 3, Panel C and FIG. 8, Panel B), the first probe and the second probe can be used to generate a reaction product to be used in downstream detection and amplification operations (e.g., dPCR, qPCR), as described elsewhere herein. The analyte can be detected using the reaction product, with ultra-sensitivity of a detection limit of about 0.1 fg/mL or less for detecting the analyte. The overall process from complex formation to analyte detection is fast, performing all operations in no more than about 90 minutes.

In another aspect, a method for analyte detection comprises (a) providing: (i) a first probe coupled to a surface of a substrate, and (ii) the analyte; (b) binding the first probe coupled to the surface of the substrate and a second probe to the analyte, to generate a complex; (c) using the first probe and the second probe of the complex to generate a reaction product; and (d) detecting the analyte using the reaction product. In some embodiments, (a)-(d) are performed in no more than about 90 minutes. In some embodiments, (a)-(d) are performed in no more than about 480 minutes, no more than about 420 minutes, no more than about 360 minutes, no more than about 300 minutes, no more than about 240 minutes, no more than about 180 minutes, no more than about 150 minutes, no more than about 120 minutes, no more than about 60 minutes, no more than about 45 minutes, no more than about 30 minutes, no more than about 15 minutes, no more than about minutes, no more than about 10 minutes, no more than about 5 minutes, or no more than about 1 minute. In some embodiments, the method comprises, prior to (c), the complex coupled to the surface of the substrate is washed at most once. In some embodiments, prior to (c), the complex coupled to the surface of the substrate is washed at most twice, at most three times, at most four times, at most five times, at most six times, at most seven times, at most eight times, at most nine times, at most ten times, or at most twenty times.

The method above describes an analyte detection method that builds a probe-analyte complex using a surface bound probe and conducting a probe reaction. For example, as shown in FIG. 3, Panels B and C and described elsewhere herein, a first probe (e.g., Probe A in FIG. 3, Panel B) coupled to a surface of a substrate and a second probe (e.g., Probe B in FIG. 3, Panel C) are bound to an analyte to generate a complex (e.g., complex as shown in FIG. 3, Panel C). The complex can be formed on the surface of the substrate (as shown in FIG. 3, Panel C). After the complex is formed, the first probe and the second probe can be used to generate a reaction product to be used in downstream detection and amplification operations (e.g., dPCR, qPCR), as described elsewhere herein. The reaction product can be generated on the same substrate used to build the probe-analyte complex. The analyte can be detected using the reaction product.

In another aspect, a method for analyte detection, comprises: (a) providing a complex coupled to a surface of a substrate, wherein the complex comprises: (i) an analyte; (ii) a first probe coupled to the analyte; (iii) a second probe coupled to the analyte, wherein the first probe or the second probe is coupled to the surface of the substrate; (b) using the first probe and the second probe to generate a reaction product; and (c) detecting the analyte using the reaction product, wherein, prior to (b), the complex coupled to the surface of the substrate is washed at most once. In some embodiments, prior to (b), the complex coupled to the surface of the substrate is washed at most twice, at most three times, at most four times, at most five times, at most six times, at most seven times, at most eight times, at most nine times, at most ten times, or at most twenty times. In some embodiments, (a)-(c) are performed in no more than about 90 minutes. In some embodiments, (a)-(c) are performed in no more than about 480 minutes, no more than about 420 minutes, no more than about 360 minutes, no more than about 300 minutes, no more than about 240 minutes, no more than about 180 minutes, no more than about 150 minutes, no more than about 120 minutes, no more than about 60 minutes, no more than about 45 minutes, no more than about 30 minutes, no more than about 15 minutes, no more than about minutes, no more than about 10 minutes, no more than about 5 minutes, or no more than about 1 minute.

The method above describes an analyte detection method that performs at most one wash prior to conducting a reaction and is agnostic to how the probe-analyte complex is formed. For example, as shown in FIG. 3, Panel C and FIG. 8, Panel B and described elsewhere herein, a complex coupled to a surface of a substate comprises an analyte, a first probe coupled to the analyte, and a second probe coupled to the analyte. The complex can be formed in solution (as shown in FIG. 8, Panel A) or on the surface of the substrate (as shown in FIG. 3, Panel C). The first probe or the second probe can be coupled to the surface of the substrate. Prior to using the first probe and the second probe to generate a reaction product, the complex coupled to the surface of the substrate can be washed at most once to remove any unbound probes and analytes (as shown in FIG. 8, Panel C). The analyte can be detected using the reaction product.

In another aspect, a method for analyte detection comprises: (a) generating a complex, wherein the complex comprises: (i) an analyte; (ii) a first probe coupled to the analyte; and (iii) a second probe coupled to the analyte; (b) without washing the complex after (a), coupling the complex generated in (a) to a surface of a substrate; (c) using the first probe and the second probe of the complex coupled to the surface of the substrate to generate a reaction product; and (d) detecting the analyte using the reaction product. In some embodiments, (a)-(d) are performed in no more than about 90 minutes. In some embodiments, (a)-(d) are performed in no more than about 480 minutes, no more than about 420 minutes, no more than about 360 minutes, no more than about 300 minutes, no more than about 240 minutes, no more than about 180 minutes, no more than about 150 minutes, no more than about 120 minutes, no more than about 60 minutes, no more than about 45 minutes, no more than about 30 minutes, no more than about 15 minutes, no more than about minutes, no more than about 10 minutes, no more than about 5 minutes, or no more than about 1 minute. In some embodiments, the method comprises, prior to (c), washing the complex coupled to the surface of the substrate at most once. In some embodiments, prior to (c), washing the complex coupled to the surface of the substrate at most twice, at most three times, at most four times, at most five times, at most six times, at most seven times, at most eight times, at most nine times, at most ten times, or at most twenty times.

The method above describes an analyte detection method that generates the probe-analyte complex in solution and couples the probe-analyte complex to a surface of a substrate without prior washing and using the same surface of the substrate to do a reaction. For example, as shown in FIG. 8, Panels A-F and described elsewhere herein, a complex is formed in solution comprising an analyte, a first probe coupled to the analyte, and a second probe coupled to the analyte (e.g., complex as shown in FIG. 8, Panel A). Without washing the complex after the complex as described is formed, the complex can be coupled to a surface of a substrate (e.g., complex capture onto surface of the substate as shown in FIG. 8, Panel B). The complex may be washed after the complex is coupled to the surface of the substrate (e.g., wash to remove any unbound species as shown in FIG. 8, Panel C). The first probe and the second probe of the complex coupled to the surface of the substate can be used to generate a reaction product (e.g., dsDNA formation as a reaction product from ligation of the probes as shown in FIG. 8, Panels D and E). The analyte can be detected using the reaction product (e.g., quantifying the dsDNA with dPCR or qPCR as shown in FIG. 8, Panel F).

In another aspect, a composition comprises a plurality of probes, wherein: (a) a first probe of the plurality of probes comprises: (1) a first binding moiety; and (2) a first biomolecule, wherein: (i) the first probe comprising the first biomolecule is coupled to a surface of a substrate, wherein a density of the first probe coupled to the surface of the substrate corresponds to no more than about 5% surface area occupancy of the surface; and (ii) the first binding moiety is configured to bind to an analyte; (b) a second probe of the plurality of probes comprises: (1) a second binding moiety; and (2) a second biomolecule, wherein the second binding moiety is configured to bind to the analyte.

The above composition describes a composition with controlled surface area occupancy of a probe coupled to a surface of a substrate and is agnostic to how the probe-analyte complex is formed. The complex can be formed in solution (as shown in FIG. 8, Panel A) or on the surface of the substrate (as shown in FIG. 3, Panel C). For example, as shown in FIG. 3, Panels A-E and FIG. 8, Panels A and B and described elsewhere herein, a first probe of a plurality of probes can comprise a first binding moiety (e.g., Probe A as shown in FIG. 3, Panel A and FIG. 8, Panel A) and a first biomolecule (e.g., Probe A Oligo as shown in FIG. 3, Panel A, oligonucleotide strand extending from Probe A in FIG. 8, Panel A), wherein the first probe comprises the first biomolecule and is coupled to the surface of the substrate (e.g., Probe A coupled to the bead surface as shown in FIG. 3, Panel B and FIG. 8, Panel B). The first probe on the surface of the substrate can be selected such that a density of the first probe coupled to the surface of the substrate corresponds to no more than about 5% surface area occupancy of the substrate, as described elsewhere herein, to minimize non-specific binding and background signals, as shown in FIG. 3, Panels D and E. The first binding moiety can be configured to bind to the analyte (e.g., Probe A configured to bind to the analyte/target protein as shown in FIG. 3, Panel B and FIG. 8, Panel A). The composition also comprises a second probe of a plurality of probes comprising a second binding moiety (e.g., Probe B as shown in FIG. 3, Panel A and FIG. 8, Panel A) and a second biomolecule (e.g., Probe B Oligo as shown in FIG. 3, Panel A, oligonucleotide strand extending from Probe B in FIG. 8, Panel A), with the second binding moiety configured to bind to the analyte (e.g., Probe B configured to bind to the analyte/target protein as shown in FIG. 3, Panel C and FIG. 8, Panel A).

In another aspect, a method of detecting an analyte comprises: (a) providing a complex coupled to a surface of a substrate, the complex comprising: (A) a first probe comprising: (1) a first binding moiety; and (2) a first biomolecule, wherein: (i) the first probe comprises the first biomolecule and is coupled to the surface of the substrate; (ii) a density of the first probe coupled to the surface of the substrate corresponds to no more than about 5% surface area occupancy of the surface; and (iii) the first binding moiety is bound to an analyte; (B) a second probe comprising: (1) a second binding moiety; and (2) a second biomolecule, wherein the second binding moiety is bound to the analyte; (b) using the first probe and the second probe to generate a reaction product; and (c) detecting the analyte using the reaction product. In some embodiments, (a)-(c) are performed in no more than about 90 minutes. In some embodiments, (a)-(c) are performed in no more than about 480 minutes, no more than about 420 minutes, no more than about 360 minutes, no more than about 300 minutes, no more than about 240 minutes, no more than about 180 minutes, no more than about 150 minutes, no more than about 120 minutes, no more than about 60 minutes, no more than about 45 minutes, no more than about 30 minutes, no more than about 15 minutes, no more than about minutes, no more than about 10 minutes, no more than about 5 minutes, or no more than about 1 minute. In some embodiments, prior to (b), washing the complex coupled to the surface of the substrate at most once. In some embodiments, prior to (b), washing the complex coupled to the surface of the substrate at most twice, at most three times, at most four times, at most five times, at most six times, at most seven times, at most eight times, at most nine times, at most ten times, or at most twenty times.

The method above describes a method to detect an analyte by controlling the surface area occupancy of a probe coupled to a surface of a substrate and is agnostic to how the probe-analyte complex is formed. The complex can be formed in solution (as shown in FIG. 8, Panel A) or on the surface of the substrate (as shown in FIG. 3, Panel C). For example, as shown in FIG. 3, Panels A-E and FIG. 8, Panels A and B and described elsewhere herein, a complex coupled to a surface of a substate comprises a first probe comprising a first binding moiety (e.g., Probe A as shown in FIG. 3, Panel A and FIG. 8, Panel A) and a first biomolecule (e.g., Probe A Oligo as shown in FIG. 3, Panel A, oligonucleotide strand extending from Probe A in FIG. 8, Panel A), wherein the first probe comprises the first biomolecule and is coupled to the surface of the substrate (e.g., Probe A coupled to the bead surface as shown in FIG. 3, Panel B and FIG. 8, Panel B). The first probe on the surface of the substrate can be selected such that a density of the first probe coupled to the surface of the substrate corresponds to no more than about 5% surface area occupancy of the substrate, as described elsewhere herein, to minimize non-specific binding and background signals, as shown in FIG. 3, Panels D and E. The first binding moiety can be bound to the analyte (e.g., Probe A configured to bind to the analyte/target protein as shown in FIG. 3, Panel B and FIG. 8, Panel A). The complex coupled to the surface of the substrate also comprises a second probe comprising a second binding moiety (e.g., Probe B as shown in FIG. 3, Panel A and FIG. 8, Panel A) and a second biomolecule (e.g., Probe B Oligo as shown in FIG. 3, Panel A, oligonucleotide strand extending from Probe B in FIG. 8, Panel A), with the second binding moiety bound to the analyte (e.g., Probe B configured to bind to the analyte/target protein as shown in FIG. 3, Panel C and FIG. 8, Panel A). The first probe and the second probe can be used to generate a reaction product to be used in downstream detection and amplification operations (e.g., dPCR, qPCR), as described elsewhere herein. The analyte can be detected using the reaction product.

In another aspect, a composition, comprises a plurality of probes, wherein: (a) a first probe of the plurality of probes comprises: (1) a first binding moiety covalently attached to a first member of a binding pair; and (2) a first nucleic acid molecule, wherein the first binding moiety is configured to bind to an analyte; (b) a surface of a substrate comprising a second member of the binding pair; and (c) a second probe of the plurality of probes comprises: (1) a second binding moiety; and (2) a second nucleic acid molecule, wherein the second binding moiety is configured to bind to the analyte.

The composition above describes a composition comprising a plurality of probes, wherein a first probe comprises a first binding moiety covalently attached to a first member of a binding pair and is agnostic to how the probe-analyte complex is formed. The complex can be formed in solution (as shown in FIG. 8, Panel A) or on the surface of the substrate (as shown in FIG. 3, Panel C). For example, as shown in FIG. 3, Panels A-C and FIG. 8, Panel B and described elsewhere herein, a composition comprising a plurality of probes, wherein a first probe of the plurality of probes comprises a first binding moiety (e.g., Probe A as shown in FIG. 3, Panel A and FIG. 8, Panel A) covalently attached to a first member of a binding pair (e.g., Probe A covalently attached to biotin as shown in FIG. 3, Panel A and FIG. 8, Panel A), and a first nucleic acid molecule (e.g., Probe A Oligo as shown in FIG. 3, Panel A, oligonucleotide strand extending from Probe A as shown in FIG. 8, Panel A), with the first binding moiety configured to bind to an analyte (e.g., Probe A configured to bind to analyte/target protein as shown in FIG. 3, Panel B and FIG. 8, Panel A). The surface of the substate comprises a second member of the binding pair (e.g., the streptavidin bead surface as shown in FIG. 3, Panel B and FIG. 8, Panel B). The composition can also comprise a second probe of the plurality of probes comprising a second binding moiety (e.g., Probe B as shown in FIG. 3, Panel A and FIG. 8, Panel A) and a second nucleic acid molecule (e.g., Probe B Oligo as shown in FIG. 3, Panel A, oligonucleotide strand extending from Probe B as shown in FIG. 8, Panel A), wherein the second binding moiety can be configured to bind to the analyte (e.g., Probe B configured to bind to analyte/target protein as shown in FIG. 3, Panel C and FIG. 8, Panel A).

In another aspect, a method of detecting an analyte comprises: (a) providing a complex on a surface of a substrate, the complex comprising: (A) a first probe comprising: (1) a first binding moiety covalently attached to a first member of a binding pair; and (2) a first nucleic acid molecule, wherein the first binding moiety is bound to an analyte; (B) the surface of the substrate comprising a second member of the binding pair, the second member of the binding pair bound to the first member of the binding pair such that the first probe is bound to the surface; and (C) a second probe comprising: (1) a second binding moiety; and (2) a second nucleic acid molecule, wherein the second binding moiety is configured to bind to the analyte; (b) using the first probe and the second probe to generate a reaction product; and (c) detecting the analyte using the reaction product. In some embodiments, the method further comprises, prior to (b), washing the complex coupled to the surface of the substrate at most once. In some embodiments, the method further comprises, prior to (b), washing the complex coupled to the surface of the substrate at most twice, at most three times, at most four times, at most five times, at most six times, at most seven times, at most eight times, at most nine times, at most ten times, or at most twenty times. In some embodiments, (a)-(c) are performed in no more than about 90 minutes. In some embodiments, (a)-(c) are performed in no more than about 480 minutes, no more than about 420 minutes, no more than about 360 minutes, no more than about 300 minutes, no more than about 240 minutes, no more than about 180 minutes, no more than about 150 minutes, no more than about 120 minutes, no more than about 60 minutes, no more than about 45 minutes, no more than about 30 minutes, no more than about 15 minutes, no more than about minutes, no more than about 10 minutes, no more than about 5 minutes, or no more than about 1 minute.

The method above describes a method to detect an analyte by providing a complex on a surface of a substrate wherein the complex has a probe comprising a first binding moiety covalently attached to a first member of a binding pair, and is agnostic to how the probe-analyte complex is formed. The complex can be formed in solution (as shown in FIG. 8, Panel A) or on the surface of the substrate (as shown in FIG. 3, Panel C). For example, as shown in FIG. 3, Panels A-C and FIG. 8, Panel B and described elsewhere herein, a complex on a surface of a substate comprises a first probe comprising a first binding moiety (e.g., Probe A as shown in FIG. 3, Panel A and FIG. 8, Panel A) covalently attached to a first member of a binding pair (e.g., Probe A covalently attached to biotin as shown in FIG. 3, Panel A and FIG. 8, Panel A), and a first nucleic acid molecule (e.g., Probe A Oligo as shown in FIG. 3, Panel A, oligonucleotide strand extending from Probe A as shown in FIG. 8, Panel A), with the first binding moiety bound to an analyte (e.g., Probe A bound with analyte/target protein as shown in FIG. 3, Panel B and FIG. 8, Panel A). The surface of the substate comprise a second member of the binding pair (e.g., the streptavidin bead surface as shown in FIG. 3, Panel B and FIG. 8, Panel B), and the second member of the binding pair bound to the first member of the binding pair such that the first probe is bound to the surface (e.g., the Probe A biotin is bound to the streptavidin bead surface bringing Probe A onto the surface as shown in FIG. 3, Panel B and FIG. 8, Panel B). The complex can also comprise a second probe comprising a second binding moiety (e.g., Probe B as shown in FIG. 3, Panel A and FIG. 8, Panel A) and a second nucleic acid molecule (e.g., Probe B Oligo as shown in FIG. 3, Panel A, oligonucleotide strand extending from Probe B as shown in FIG. 8, Panel A), wherein the second binding moiety can be configured to bind to the analyte (e.g., Probe B configured to bind to the analyte/target protein as shown in FIG. 3, Panel C and FIG. 8, Panel A). The first probe and the second probe can be used to generate a reaction product to be used in downstream detection and amplification operations (e.g., dPCR, qPCR), as described elsewhere herein. The analyte can be detected using the reaction product.

Without being bound by theory and as understood by a person skilled in the art, a fluorescence resonance energy transfer (FRET) assay can be applied to optical microscopy and can permit determination between two molecules within a distance sufficiently close for molecular interactions to occur. The mechanism of FRET can describe an energy transfer between a plurality of light-sensitive molecules or chromophores. In some embodiments, a donor fluorophore in an excited electronic state may transfer its excitation energy to a nearby acceptor chromophore in a non-radiative fashion through long-range dipole-dipole interactions. FRET can be an accurate measurement of molecular proximity at angstrom distances and can be highly efficient if the donor chromophore and the acceptor chromophore are positioned within the distance at which half the excitation energy of the donor can be transferred to the acceptor chromophore. In some embodiments, the first binding moiety may be a donor chromophore. In some embodiments, the first binding moiety may be an acceptor chromophore. In some embodiments, the second binding moiety may be a donor chromophore. In some embodiments, the second binding moiety may be an acceptor chromophore.

Without being bound by theory and as understood by a person skilled in the art, a colocalization reaction may be a technique used to determine the spatial overlap or proximity of two or more target molecules within a biological or chemical system, at times at the subcellular level. The assay may rely on the detection and analysis of two or more distinct signals, often from different fluorescent probes or markers, to assess whether the targets are localized in the same or adjacent regions of a sample. Colocalization may provide valuable insight into molecular interactions, cellular processes, or the functional organization of cellular compartments. In another embodiment, a colocalization reaction may utilize signals from two different fluorophores (or other markers) visualized and quantified in a single sample. When two molecules of interest are spatially located in the same subcellular compartment, their corresponding fluorescence signals will overlap or colocalize in the imaging system. The extent of this colocalization can be quantitatively assessed by various computational or statistical methods, allowing the researcher to determine if the two targets are likely interacting or in close proximity.

Without being bound by theory and as understood by a person skilled in the art, reaction sensitivity or detection limit may refer to the ability of an assay or reaction to detect small amounts or low concentrations of a target. For example, it can describe how responsive the system is to changes in the presence or concentration of the target molecule. In the context of biochemical, molecular, or analytical assays, sensitivity can indicate how well the assay can detect low levels of the analyte (e.g., target molecule) and distinguish it from background noise or non-target signals. Reaction sensitivity can quantify how effectively a reaction can detect the target under varying conditions, such as, when the target is present in low amounts. In some embodiments, reaction sensitivity may be expressed in terms of the detectable concentration of the target (e.g., femtogram per milliliter (fg/mL), picogram per milliliter (pg/mL)), in terms of the detection limit or limit of detection (LOD) of the target corresponding to the lowest quantity or concentration of a component that can be distinguished from the absence of that component (e.g., a blank value) with a stated confidence level (e.g., femtogram per milliliter (fg/mL), picogram per milliliter (pg/mL), molar units), in terms of in terms of the minimum detectable signal above background noise (e.g., fluorescence units, absorbance units), in terms of the strength of the signal relative to the background noise (e.g., signal-to-noise ratio), or in terms of how many molecules or particles can detect in a given volume (e.g., particles/mL). In some embodiments, concentration can be measured as mass of molecules detected per unit of volume. In some embodiments, the detection limit or limit of detection (LOD) corresponds to the lowest quantity or concentration of a component that can be reliably detected with a given analytical method. For example, a detection limit of about 0.1 fg/mL or less corresponds to reliably detecting a component with concentration of 0.1 fg/mL or less.

In some embodiments, the method comprises detecting the analyte using the reaction product, with a detection limit of about 0.1 fg/mL or less for detecting the analyte. In some embodiments, the method comprises detecting the analyte using the reaction product, with a detection limit of about 0.0000001 fg/mL or less, about 0.000001 fg/mL or less, about 0.00001 fg/mL or less, 0.0001 fg/mL or less, about 0.0005 fg/mL or less, about 0.001 fg/mL or less, about 0.005 fg/mL or less, about 0.01 fg/mL or less, about 0.02 fg/mL or less, about 0.03 fg/mL or less, about 0.04 fg/mL or less, about 0.05 fg/mL or less, about 0.06 fg/mL or less, about 0.07 fg/mL or less, about 0.08 fg/mL or less, about 0.09 fg/mL or less, about 0.1 fg/mL or less, about 0.2 fg/mL or less, about 0.3 fg/mL or less, about 0.4 fg/mL or less, about 0.5 fg/mL or less, about 0.6 fg/mL or less, about 0.7 fg/mL or less, about 0.8 fg/mL or less, about 0.9 fg/mL or less, about 1 fg/mL or less, about 2 fg/mL or less, about 5 fg/mL or less, about 10 fg/mL or less, about 15 fg/mL or less, about 20 fg/mL or less, about 25 fg/mL or less, about 30 fg/mL or less, about 40 fg/mL or less, about 50 fg/mL or less, about 100 fg/mL or less, about 200 fg/mL or less, about 400 fg/mL or less, about 500 fg/mL or less, about 600 fg/mL or less, about 800 fg/mL or less, about 900 fg/mL or less, about 1000 fg/mL or less, about 1.5 pg/mL or less, about 2 pg/mL or less, about 3 pg/mL or less, about 4 pg/mL or less, about 5 pg/mL or less, about 7.5 pg/mL or less, about 10 pg/mL or less, about 50 pg/mL or less, about 100 pg/mL or less, about 200 pg/mL or less, about 300 pg/mL or less, about 400 pg/mL or less, about 500 pg/mL or less, or about 1000 pg/mL or less for detecting the analyte. Without being bound by theory and as understood by a person skilled in the art, sensitivity may be measured as the mass of molecules detected per unit of volume, such as femtogram per milliliter (fg/mL) and picogram per milliliter (pg/mL).

Without being bound by theory and as understood by a person skilled in the art, reaction specificity may refer to the ability of an assay or reaction to selectively detect the target molecule while minimizing the detection of non-target molecules or background signals. For example, specificity can measure how well an assay distinguishes the target from other substances (e.g., other similar molecules, contaminants, noise) that may be present in a sample. High specificity (e.g., higher %) may mean the assay can discriminate between the target and other potential interfering substances, ensuring that the detected signal is due to the actual target molecule, not other molecules or nonspecific interactions. In some embodiments, reaction specificity may be expressed in terms of the portion of true target detections out of all actual positive samples (e.g., a true positive rate, a percentage), in terms of the strength of the signal from the target molecule to the background noise from non-target molecules (e.g., signal-to-noise ratio), in terms of the degree to which the assay binds to molecules other than the intended target (e.g., a percentage cross-reactivity), in terms of how much of the surface area or binding sites are occupied by the target versus non-target molecules (e.g., a percentage occupancy, a percentage binding).

In some embodiments, the method comprises detecting the analyte using the reaction product with a specificity of greater than about 75%. In some embodiments, the method comprises detecting the analyte using the reaction product with a specificity of greater than about 50%. In some embodiments, the method comprises detecting the analyte using the reaction product with a specificity of greater than about 10%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or greater than about 99%. Without being bound by theory and as understood by a person skilled in the art, specificity may be measured as a percentage indicating the proportion of correctly identified target interactions out of all interactions, including non-targets (e.g., about 50% specificity, about 75% specificity).

In some embodiments, the method can further comprise amplifying complementary nucleic acid molecule of the ligated nucleic acid molecule, thereby generating a reaction product. In some embodiments, the amplifying comprises performing a polymerase chain reaction. In some embodiments, the polymerase chain reaction is a quantitative polymerase chain reaction. Without being bound by theory and as understood by a person skilled in the art, quantitative polymerase chain reaction (qPCR) enables the amplification and quantification of specific nucleic acids (e.g., DNA, RNA) in a sample. qPCR allows for the detection and quantification of specific nucleic acid sequences during the amplification process in real time. Fluorescent dyes or probes can emit light upon binding to the amplified DNA. In some embodiments, the probe is cleaved, releasing the fluorescent molecule, which provides a quantitative measure of the target nucleic acid in the sample. In some embodiments, the probe is not cleaved but still generates a fluorescent signal, providing a quantitative measure of the target nucleic acid in the sample. The fluorescence signal can be generated through binding-induced conformational changes. The core components of qPCR may include the DNA or RNA template, primers, probes, polymerase enzyme, dNTPs, and a fluorescent detection system. In a qPCR reaction, the target DNA or cDNA (for RNA) may be amplified through a series of thermal cycles. During each cycle, the fluorescence emitted by the dye or probe increases in proportion to the amount of amplified product, and this fluorescence is continuously monitored throughout the reaction. The level of fluorescence is directly related to the amount of product formed, and by measuring the fluorescence at each cycle, qPCR enables the calculation of the initial quantity of the target nucleic acid in the sample. The amount of target nucleic acid is determined by comparing the fluorescence of the sample to a standard curve or through analysis of the cycle threshold (Ct), which is the cycle at which the fluorescence exceeds a set threshold. The lower the Ct value, the greater the amount of initial target nucleic acid in the sample. qPCR can be performed using either intercalating dyes (such as SYBR Green) or sequence-specific probes (such as TaqMan probes), which allow for specific targeting of the desired nucleic acid sequence. Additionally, qPCR can be performed in a multiplex format, where multiple targets are quantified in the same reaction using different fluorescent dyes, enabling high-throughput analysis of several genes or sequences simultaneously.

In some embodiments, the polymerase chain reaction is a digital polymerase chain reaction. Without being bound by theory and as understood by a person skilled in the art, digital polymerase chain reaction (dPCR) enables quantifying nucleic acids by partitioning a sample into numerous individual reactions, each of which undergoes independent amplification. dPCR provides absolute quantification of target sequences without the use for standard curves or reference samples. In dPCR, the sample may be partitioned into a plurality of individual reactions, often in a microfluidic chip or a droplet-based system. These partitions may contain either zero or one copy of the target nucleic acid, allowing for precise counting of positive amplification events. The process may begin by distributing the nucleic acid sample into multiple reaction wells or droplets, followed by PCR amplification. After amplification, each partition may be analyzed to determine whether it contains a detectable signal (positive for the target) or not (negative). The number of positive partitions may be counted, and based on this count, the absolute number of target molecules in the original sample may be calculated using statistical methods. This partitioning and counting mechanism may allow dPCR to achieve a high degree of sensitivity and accuracy, even in the presence of low-abundance targets or in complex samples. An advantage of dPCR may be its ability to detect rare genetic events with high precision, making it ideal for applications that require high sensitivity, such as detecting mutations, quantifying rare alleles, measuring viral loads, or assessing gene expression levels. dPCR may also be beneficial when there is no known reference or standard curve available for quantification, as it directly measures the target sequence without relying on external calibration.

In some embodiments, the method comprises sequencing the reaction product. Without being bound by theory and as understood by a person skilled in the art, sequencing refers to the process of determining the precise order of nucleotides (adenine [A], cytosine [C], guanine [G], and thymine [T]) in a DNA or RNA molecule, providing insights into genetic information. The sequencing process may involve several operations: (1) sample preparation, where DNA or RNA is extracted, fragmented, and prepared with adapters or primers for amplification; (2) amplification and sequencing, where the DNA is read by the sequencing platform through various methods such as fluorescence or electrical signals; and (3) data analysis, where the raw sequence data is processed, aligned to reference genomes, or assembled de novo, followed by variant calling or gene expression analysis. Various sequencing methods exist, such as Sanger sequencing, Next-Generation Sequencing (NGS), and third-generation sequencing technologies such as Oxford Nanopore and PacBio sequencing. For example, Sanger sequencing involves chain-termination PCR and fluorescence detection to determine the sequence of short DNA fragments. In another example, NGS technologies enable the parallel sequencing of millions of DNA fragments in a single run, providing high-throughput and cost-effective solutions for sequencing entire genomes or transcriptomes. In another example, third-generation sequencing methods, such as PacBio and Oxford Nanopore, are capable of generating long reads, allowing for more accurate genome assembly and the detection of complex structural variants. In some embodiments, sequencing is conducted on the fluorescently labeled substrate. Sequencing on a fluorescently labeled substrate may refer to a sequencing method where the nucleic acid template, primers, or reaction products are immobilized on a solid surface or substrate that is fluorescently labeled or capable of detecting fluorescent signals during the sequencing process. For example. The nucleic acid template to be sequenced may be first immobilized on a solid substrate. The surface may be coated with a material such as a glass slide or a chip made of silicon or another biocompatible material. This surface can be chemically modified to attach or capture the DNA strands or fragments. Fluorescently labeled nucleotides, which each correspond to one of the four bases (adenine [A], cytosine [C], guanine [G], and thymine [T]), may be added to the sequencing reaction. These nucleotides are tagged with different fluorescent dyes that emit specific wavelengths of light when excited by a laser. As the sequencing reaction progresses, the DNA polymerase synthesizes complementary DNA strands from the immobilized template. During each cycle of the reaction, the polymerase can incorporate one of the fluorescently labeled nucleotides into the growing strand, and the incorporation event can be captured. The immobilized substrate may be scanned or imaged by a fluorescence detection system. For example, a laser may excite the fluorescent labels attached to the incorporated nucleotides, causing them to emit light. A camera or detector can capture the emitted fluorescence, identifying the specific nucleotide that was added to the growing DNA strand. Different fluorescent dyes can correspond to different nucleotides (e.g., red for A, green for T, blue for C, and yellow for G). The fluorescence signals detected at each operation may be analyzed by a software program, which correlates the color emitted by each nucleotide with the specific base. The sequence of emitted signals can be assembled to form the DNA sequence of the template.

In one aspect, the compositions and methods may be used to detect blood-based biomarkers to identify individuals with Alzheimer's Disease. Alzheimer's Disease (AD) is a complex, pervasive neurodegenerative disorder hallmarked by progressive damage to the cells in the brain. The pathological changes of this disease are generally characterized by accumulations of the proteins, amyloid beta (Aβ) and tau tangles. AD accounts for 60-80% of all dementia cases, with the number of individuals suffering from dementia projected to nearly triple to 139 million by 2050. This rise poses a significant public health challenge with profound implications for healthcare costs, caregiver burden, and the overall economy. Despite extensive research, a major challenge in AD research and clinical practice is the lack of easily accessible, sensitive, and accurate diagnostic tools.

Blood-based biomarkers (BBM) can accurately identify individuals with AD, highlighting their significant potential for revolutionizing AD diagnostics. Phosphorylated tau (p-Tau) species, including p-Tau 217, and Amyloid Beta (e.g., Aβ42) are biomarker candidates for AD due to their superior diagnostic accuracy and disease specificity.

Recognized herein is a need for a clinical implementation of a workflow that provides sensitive, absolute quantitation of blood-based biomarkers as analytes that may influence AD diagnostics by providing a scalable, minimally invasive, and cost-effective tool for early detection and monitoring of the disease. The ability to make absolute measurements with a workflow by controlling the substrate surface area occupancy, as described elsewhere herein, and without reliance on a standard curve (e.g., using a digital polymerase chain reaction) may significantly reduce the variability that has made reproducibility a challenge for many technologies. The disclosed compositions and methods for detecting analytes aim to demonstrate the technical feasibility of a digital proximity ligation assay platform (e.g., proximity ligation assay with digital polymerase chain reaction) to enable superior quantitation of a biomarker, including for example, p-Tau 217 and Aβ42, in plasma and serum samples with a simple, accessible workflow.

In one embodiment, a prototype p-Tau 181 assay performs on par with on-market assays. A probe construction with a first probe comprising an antibody conjugated with both a first nucleic acid strand (e.g., oligonucleotide) and biotin, and a first probe comprising an antibody conjugated with a second nucleic acid strand (e.g., oligonucleotide), as disclosed elsewhere herein, and with a workflow with a set surface area occupancy of probes on the substrate surface can achieve a lower limit of detection (LLOD) (e.g., <100 fg/mL). The findings from the prototype can be used for the development of a more comprehensive assay panel including both p-Tau 217 and Aβ42. The assay platform can leverage the disclosed composition and method for detecting an analyte with novel implementation of proximity ligation assay (PLA) using specifically designed antibody probes. When combined with digital PCR, the chemistry can enable ultra-sensitive absolute quantitation.

This technology may be expanded to a broader range of protein targets, making it a versatile tool in protein-based diagnostics. By addressing the critical features for accessibility, sensitivity, and accuracy in AD diagnostics, this digital PLA platform represents a significant advancement in AD diagnostics with the potential to significantly improve how AD is detected and managed in clinical practice.

The disclosure provides a platform for measuring AD biomarkers in blood, with the potential to transform AD diagnosis and prognosis. The compositions and methods disclosed herein introduce an approach to protein biomarker quantification, characterized by (1) reproducible and reliable results, (2) ultrasensitivity and precision to measure subtle variations, and (3) scalable and expandable technology.
(1) Reproducible and Reliable Results: Digital output for measurements without a standard curve: The compositions and methods may leverage digital PCR (dPCR). Compared to qPCR, dPCR provides absolute quantification by amplifying a complementary sequence of a ligated nucleic acid template as disclosed elsewhere herein, and by directly counting the number of target molecules, eliminating the use for a reference standard curve. This can enable consistent quantification by reducing a source for variability. Example streamlined workflow: The technology can be designed with a straightforward workflow, reducing the complexities that often cause variability in assay performance. In some embodiments, sample and antibody probes may be incubated together to form immunocomplexes. In some embodiments, immunocomplexes may be selectively captured with magnetic beads and unbound probes may be washed away to reduce background. In some embodiments, digital PCR may be used to quantify the protein concentration. Novel Dual Antibody Conjugation Technique: The chemistry may involve chemically modifying antibodies. To promote consistent performance across runs and batches, different chemistries may be used for a dual antibody conjugation technique. In some embodiments, SiteClick technology may be used to attach oligonucleotides to the two N-linked glycan sites on the Fc region of IgG antibodies, preserving their functionality. In some embodiments, the antibody probes may be biotinylated using EZ-Link Sulfo-NHS-LC-Biotin. The biotin may be smaller (e.g., 2-3 orders of magnitude) than the oligonucleotide, minimizing the risk of negatively impacting antibody performance to enhance assay reproducibility and lot-to-lot consistency.
(2) Ultrasensitivity and Precision: Spatial dilution for enhanced sensitivity: As described elsewhere herein, the compositions and methods may use a spatial dilution technique that selectively reduces background noise without compromising the signal from target proteins. While magnetic beads may be used to wash away unbound probes, some background signal generating probes may non-specifically bind to the beads. By carefully controlling the antibody-to-surface area ratio (e.g., controlling the density of the probe on substrate surface to correspond to a certain percentage surface area occupancy), the background signal can be minimized to enable reliable detection of critical biomarkers. This can be important for detecting low-abundance biomarkers in complex matrices like serum and plasma. Digital output for enhanced precision: In some embodiments, subtle sample differences may be detected by a dPCR readout. By directly counting the number of molecules present, the output of dPCR is an absolute quantification. Ultrasensitivity may be helpful in diagnostics, and this precision to measure small differences reliably can enable longitudinal monitoring for disease prognosis and staging.
(3) Scalable, Accessible, and Expandable Technology: Scalable and Accessible Technology: The assay may not be instrument-dependent. For example, it may be read on different PCR instruments. This makes the assay flexible such that the compositions and methods do not require a specific readout technology and can be scalable and accessible to a broad range of healthcare settings. Expandable Assay Catalogue: As described herein, the platform may be designed for easy expansion to a variety of diseases that can be identified with biomarkers. The specificity of the assay may be determined by the antibodies used, making it highly adaptable. For example, alpha-synuclein (a-synuclein) for Parkinson's Disease or TDP-43 for frontotemporal dementia can be seamlessly incorporated into the assay.

The compositions and methods described elsewhere herein can be useful in evaluating specificity and antibody-to-target cross-reactivity of antibody pairs, and evaluating sensitivity of antibody pairs. Each antibody in an antibody pair may be a first probe or a second probe in a composition comprising a plurality of probes, as described elsewhere herein. Each antibody in an antibody pair may be a first probe or a second probe to use in a method for detecting an analyte comprising binding a plurality of probes and conducting a reaction between the first probe and the second probe, as described elsewhere herein. In some embodiments, a plurality of antigens can be used to evaluate the degree of nonspecific binding to antibody pairs. For p-Tau 217, phosphorylated variants, including p-Tau 181 and unphosphorylated Tau isoforms, may be used. The concentrations for unphosphorylated Tau and Tau proteins phosphorylated at other sites may be in the range of tens of picograms per milliliter of protein (e.g., assay target of <1 fg/mL). For Aβ42, amyloid peptides, including Aβ38 and Aβ40, can be used. The concentrations for amyloid beta peptides may be in the range of hundreds of picograms per milliliter of protein (e.g., assay target of <100 fg/mL).

The compositions and methods described elsewhere herein may be useful in testing sensitivity of antibody pairs. Each antibody in an antibody pair may be a first probe or a second probe in a composition comprising a plurality of probes, as described elsewhere herein. Each antibody in an antibody pair may be a first probe or a second probe to use in a method for detecting an analyte comprising binding a plurality of probes and conducting a reaction between the first probe and the second probe, as described elsewhere herein. In some embodiments, the lower limit of detection (LLOD) and lower limit of quantitation (LLOQ) of each antibody pair (e.g., a first probe and a second probe, p-Tau 217 and Aβ42) may identified by quantitating a serial dilution using the antibody pairs under identical conditions. In some embodiments, a low end of the dilution series for p-Tau 217 may be about 0.1 fg/mL and a low end of the dilution series for Aβ42 is about 10 fg/mL.

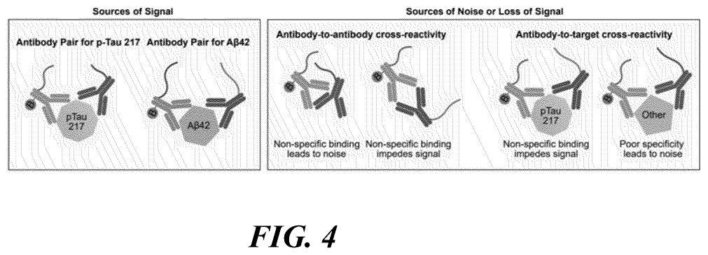

The compositions and methods described elsewhere herein may be useful to evaluate the background signal level, as shown in FIG. 4, produced by the antibody pairs. In some embodiments, lower starting background signal may provide a better starting point for developing a highly sensitive assay. Lot-to-lot consistency may be a factor to ensure long-term reliability and viability of the antibody for assay production. The cross-reactivity, LLOD and background signal levels may be compared between lots of antibodies for each candidate pairs.

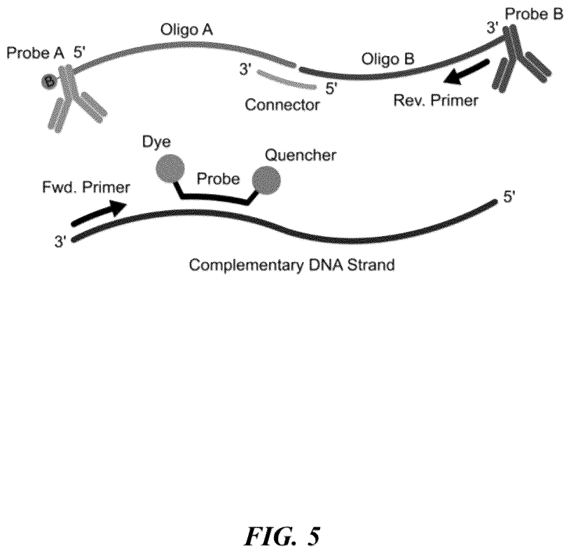

The compositions and methods described elsewhere herein may be useful in performing a proximity ligation assay. In one embodiment, a pair of probes (e.g., Probe A and Probe B) may be selected, with each probe comprising a binding moiety (e.g., Probe A comprising antibody A, Probe B comprising antibody B) each coupled to a biomolecule (e.g., unique oligonucleotide sequence Oligo A for Probe A and unique oligonucleotide sequence Oligo B for Probe B), as shown in FIG. 5. The two antibodies may bind to a target protein in a sample, the attached oligonucleotide strands may be brought into close proximity. The oligonucleotides on pairs of antibodies brought in proximity due to antigen recognition, may hybridize to a connector oligonucleotide so that the antibody-conjugated oligonucleotide may be joined by enzymatic ligation by a ligase, as described elsewhere herein. In some embodiments, the concentration of the connector oligo that enables ligation may impact the signal level of the target. In some embodiments, the length of the conjugated sequence, location of the primer/probe, and the melt temperature of the oligonucleotides may be aligned to ensure consistent and accurate quantitation. A sample-antibody mixture may be added to a PCR master mix containing ligase to ligate the first biomolecule (e.g., Oligo A) and the second biomolecule (e.g., Oligo B), thereby generating a ligated nucleic acid template across the first probe (e.g., Probe A) and the second probe (e.g., Probe A), as described elsewhere herein. The enzyme may ligate the two adjacent DNA strands and create a nucleic acid molecule (e.g., a single continuous DNA strand). The single continuous DNA strand may be amplified through dPCR or qPCR, to provide quantification of the target protein's abundance in the sample, as described elsewhere herein The complementary strand may be synthesized after the first round of PCR.

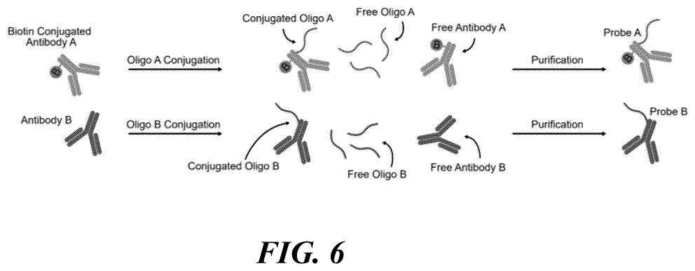

The systems and methods disclosed herein may be useful in performing oligonucleotide-probe conjugation and purification. In some embodiments, the conjugation reaction may result in a mixture of species—antibodies with an oligo attached, antibodies with no oligo, and free floating oligos, as shown in FIG. 6. Antibodies with no oligo may serve as a competitive blocker while free oligos may increase background signal levels.

The compositions and methods disclosed herein may be adapted on an automated liquid handler. In one embodiment, the disclosed assay may be adapted onto an automated liquid handling workstation to process up to 96 samples per batch to include samples, controls, and replicates, and automate the preparation, incubation, washing, and extraction operations of the digital immunoassay.

In some embodiments, the reagent preparation and incubation operations may be transferred onto the workstation. In some embodiment, the dilution of probe mixtures using probe buffer may be automated, the dilution of sample using sample buffer may be automated, the mixing diluted sample with probe solution and may be automated, or the incubation to allow immunocomplex formation may be automated.

In some embodiments, the automation may reduce variation, and allow for more accurate control of incubation times. In some embodiments, the automation may be adapted in stages.

In some embodiments, the magnetic bead capture, wash, and extraction operations may be automated. In some embodiments, the addition of magnetic streptavidin-coated beads into the immunocomplex solution may be automated, the incubation to bind immunocomplexes to beads may be automated, the beads washing using wash buffer may be automated, or extraction of dsDNA for dPCR may be automated.

### Computer Systems

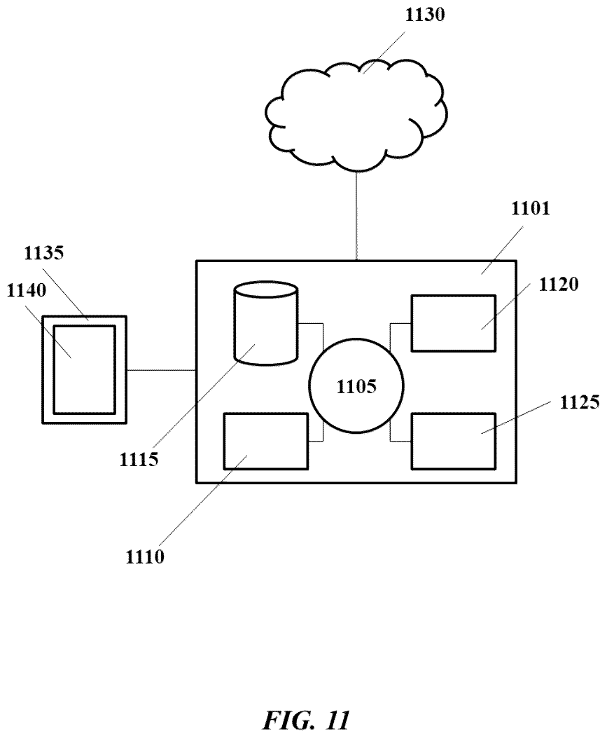

The present disclosure provides computer systems that are programmed to implement methods of the disclosure. FIG. 11 shows a computer system 1101 that is programmed or otherwise configured to performed the methods disclosed herein. The computer system 1101 can regulate various aspects of the preparation, incubation, washing, extraction procedures of the colocalization reactions and digital immunoassays of the present disclosure, such as, for example, reaction conditions, flow conditions, reagent conditions, dilution conditions of probe mixtures and sample mixtures. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., a personal computer, a Smart phone). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, sample conditions, reaction conditions, incubation conditions. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, calculate the sufficient distance for a first biomolecule and a second biomolecule to participate in a reaction with one another.

### EXAMPLES

### Example 1. Demonstration of the Effect of Controlling Probe-Substrate/Bead Surface Area Occupancy

As described herein, it may be beneficial to characterize the difference in background/non-specific signals with different amount of beads present. In one example, a commercially available PLA antibody pair and Protein G coated magnetic beads were used to demonstrate the effect of controlling probe-substrate/bead surface area occupancy, to reduce the likelihood of nonspecific binding resulting in probe colocalization. Protein G beads were designed to capture antibodies and bring the two antibody probes onto the bead surface. In the experiment, the two probes (e.g., antibodies) were incubated with beads in the absence of target molecules. Without any target, the signal generated was from unintended colocalizations of the two antibodies when in close proximity, similar to background signals in a normal assay. The goal was to demonstrate the effect of controlling probe-substrate surface area occupancy to reduce the number of colocalizations by increasing the available surface area. Surface area occupancy (%) was determined by first multiplying the surface area covered by each probe by the number of probes, then dividing by the substrate surface area, e.g., [Surface Area of Each Probe x Number of Probes]/[Substrate Surface Area]. The surface area of each probe can be influenced by the size of each probe. Four different probe-to-substrate/bead ratios were created by holding the probe numbers constant while varying the number of Protein G capture beads. Table 1 shows the bead numbers used and the average substrate surface occupation and probe number per bead with ˜5 billion antibody pairs.

TABLE 1
Capture Bead Numbers and Respective Substrate Surface
Occupation and Average Probe Number per Bead

| Capture | | Average Probe |
|---|---|---|
| Bead | Average Substrate Surface | Number |
| Number | Occupation (%) | per Bead |
| 1M | ~1/60 occupation (~1.67%) | Avg of 5,000 Probes |
| 10M | ~1/600 occupation (~0.167%) | Avg of 500 Probes |
| 100M | ~1/6,000 occupation (~0.0167%) | Avg of 50 Probes |
| 1B Beads | ~1/60,000 occupation (~0.00167%) | Avg of 5 Probes |

In this example, four technical replicates were created for each bead number condition with four technical replicates quantified for each condition for a total of eight replicates per bead number. The beads were incubated with the antibody pairs overnight, washed three times and subsequently analyzed using qPCR, as described elsewhere herein. Table 2 below shows the values reported were qPCR Ct values that quantify the amount of background signal produced as the result of non-specific co-localization. Lower cycle thresholds (Cts) indicated higher signal.

TABLE 2
Capture Bead Number vs. Cycle Thresholds

| Bead | | Ligation | Ct | Ct | L | L |
|---|---|---|---|---|---|---|
| Number | Ct | Event | avg | Stdev | avg | Stdev |
| 1B | 25.48 | 5.88E+03 | 25.53 | 0.26 | 5.74E+03 | 1.01E+03 |
| | 25.85 | 4.54E+03 | | | | |
| | 25.56 | 5.55E+03 | | | | |
| | 25.23 | 7.00E+03 | | | | |
| 100M | 22.38 | 5.04E+04 | 22.37 | 0.03 | 5.08E+04 | 1.18E+03 |
| | 22.39 | 5.02E+04 | | | | |
| | 22.39 | 4.99E+04 | | | | |
| | 22.32 | 5.25E+04 | | | | |
| 10M | 19.73 | 3.16E+05 | 19.72 | 0.03 | 3.19E+05 | 7.59E+03 |
| | 19.74 | 3.15E+05 | | | | |
| | 19.67 | 3.30E+05 | | | | |
| | 19.74 | 3.15E+05 | | | | |
| 1M | 17.84 | 1.17E+06 | 17.85 | 0.02 | 1.17E+06 | 1.41E+04 |
| | 17.85 | 1.17E+06 | | | | |
| | 17.87 | 1.15E+06 | | | | |
| | 17.83 | 1.18E+06 | | | | |

The results indicated that the Ct values associated with the higher bead condition were larger, suggesting fewer co-localization events. With higher bead numbers, there were fewer co-localization events per bead basis, and in total. The results hold when normalized, e.g., comparing the same numbers of beads. The number of ligation events based on the Ct may be calculated from the formula: Ligation events=2^{(38-Ct)}

According to this equation, a Ct value of 38 represents a single ligation event occurred from the colocalization of the two probes; the Ct value obtained from qPCR may be used to calculate number ligations (hence colocalizations).

In this example, as shown in Table 3, on average, the 1 million bead condition had total of 2^{(38-17.85)} or ˜1.17 million co-localization events per 1 million beads. The 1 billion beads condition had a total of ˜5,700 co-localization events or about 5.7 per 1 million beads.

TABLE 3
Normalized Ligation Number per 1 Million Beads

| Bead Number | Avg Ligations per 1 million beads |
|---|---|
| 1B | 5.74E+00 |
| 100M | 5.08E+02 |
| 10M | 3.19E+04 |
| 1M | 1.17E+06 |

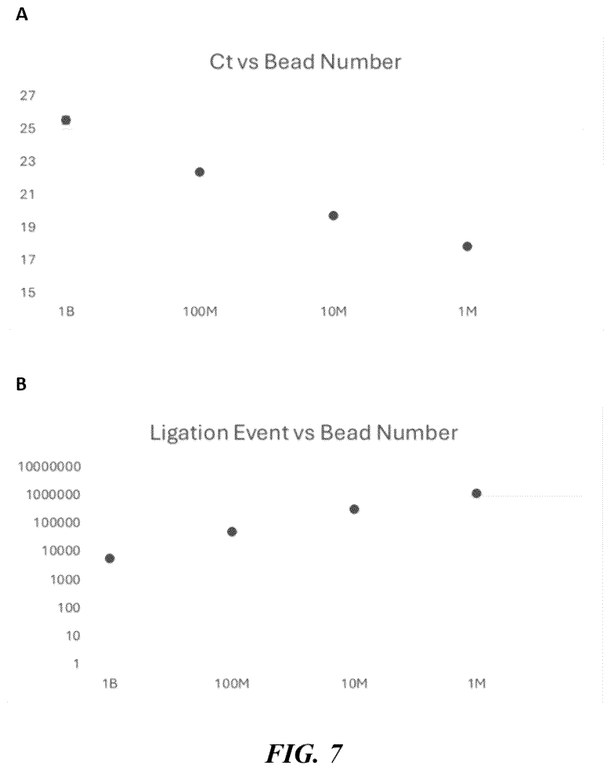

As shown in FIG. 7, Panels A and B, Ct values were larger (e.g., fewer colocalizations) when more beads were used (e.g., FIG. 7, Panel A), therefore fewer total colocalization or ligation events occurred (e.g., FIG. 7, Panel B). FIG. 7, Panel A shows the qPCR quantitation result (Ct) versus number of capture beads with same number of antibody pair. FIG. 7, Panel B shows the ligation events versus number of capture beads. The results show the number of ligation events may be controlled by surface area.

The results demonstrated with more surface area available (due to the presence of higher capture bead number), the signal generated by unintended surface co-localization may be mitigated by spatially diluting out the background signal.

### Example 2. PLA Assay Workflow

In another example, a prototype assay was created to demonstrate the ability to deliver accessible, sensitive and accurate tests. FIG. 8, Panels A-F outline the general operations of a PLA assay and the molecular interactions occurring. In the first operation, the sample was incubated with two probes to form the immunocomplex in solution, as shown in FIG. 8, Panel A and described elsewhere herein. Probe A comprises an antibody conjugated with both a first nucleic acid strand (e.g., oligonucleotide) and biotin, while Probe B was conjugated with a second nucleic acid strand (e.g., oligonucleotide) (FIG. 8, Panel A). The immunocomplex was incubated with a suspension of streptavidin-coated magnetic beads to specifically capture the immunocomplex (FIG. 8, Panel B). The beads were then washed to remove unwanted material including unbound probes and any contamination substance from the sample, retaining the immunocomplex (FIG. 8, Panel C). The immunocomplex may be incubated with a ligase to ligate the two oligonucleotides on the two probes to form a ligated nucleic acid template (FIG. 8, Panel D). The ligated nucleic acid template may be thermocycled with polymerase to inactivate the ligase and generate a complementary sequence of the ligated nucleic acid template and amplified to generate the double-stranded DNA (dsDNA) (e.g., a nucleic acid molecule) released from the immunocomplex (FIG. 8, Panel E). The dsDNA may be quantified by either qPCR or dPCR (FIG. 8, Panel F). For dPCR, the dsDNA is captured in the supernatant and further partitioned and quantified in dPCR. For qPCR, the dsDNA may be quantified and measured while remaining bound onto the immunocomplex. This assay workflow can be applied to measure pTau-181 levels in serum/plasma samples with a simple workflow that leverages spatial dilution.

### Example 3. Digital PLA

In one example, digital PCR was used to demonstrate a digital PLA workflow. Leveraging digital quantitation can allow the assay to provide absolute quantification without relying on standard curves that depend on reference materials.

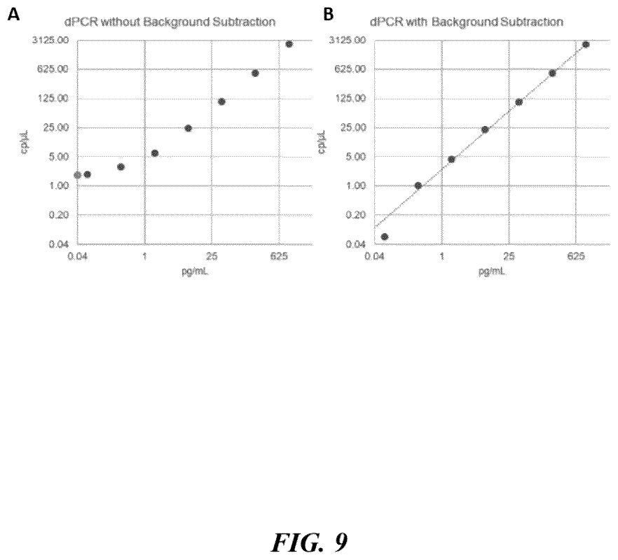

In one example, reference material from a commercially available p-Tau 181 assay kit was used to demonstrate the ability to provide absolute quantification. A 7-point standard curve with 5-fold dilutions was created as per the vendor instructions from 1000 pg/mL down to 0.064 pg/mL, and the entire standard curve was processed using the assay workflow as described above. The ligated DNA was then quantified using the Absolute Q Digital PCR system (FIG. 9, Panel A). Another advantage of utilizing digital quantitation may be the ability to directly subtract out background signals. The left most dot in FIG. 9, Panel A indicated the background signal level and the 7th dilution point (0.064 pg/mL) cannot be distinguished from the background and curve had lost linearity at the 6^{th} point. However, if the background signal was subtracted from the quantitation results, linearity is restored down to the 6th point—0.32 pg/mL (FIG. 9, Panel B). The results demonstrated sub-pg/mL quantitation of a reference material using dPCR and with continued optimization the LLOD may be improved by at least 10-fold.

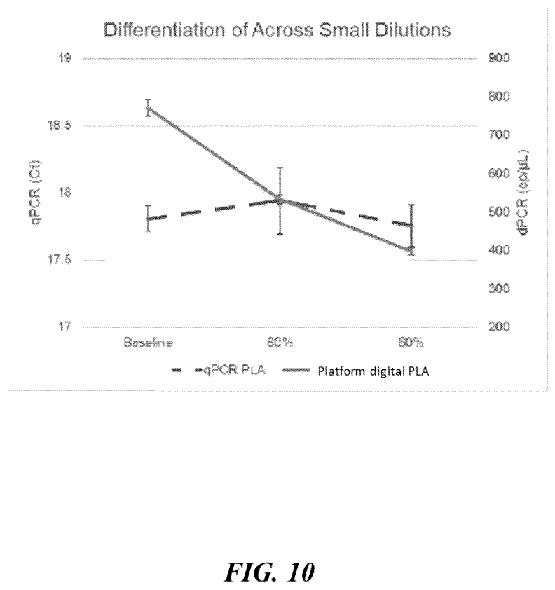

With the precise quantitation of digital PCR, the assay workflow can detect small changes, as shown in FIG. 10. This feature can improve longitudinal monitoring to measure disease progression and, if a therapeutic is making an impact. In one example, standard material were used to create dilutions that simulate a small change in the target protein. To achieve this, a baseline concentration was selected and a dilution curve consisting of small changes were made. The baseline sample was diluted by 20% and 40%—with expected concentrations to be 80% and 60% of the baseline, respectively. FIG. 10 summarizes the results using a homogenous PLA run on qPCR compared to the disclosed platform workflow run on dPCR with error bars indicating standard deviation of triplicates. qPCR technology may be inherently limited-it may not easily distinguish small changes and had much larger variation within the measurements. Thus, it may not be possible to statistically distinguish the difference between the dilutions. In contrast, the dPCR-based platform assay had much better consistency between the replicates, and it may be possible to distinguish the different dilutions with statistical significance (p-value<0.01 between each condition). As disclosed elsewhere herein, a prototype assay using background-reducing technology was created. The platform assay may achieve high analytical performance with a simple workflow enabled by (1) spatial dilution (e.g., adjusting the ratio between probes and substrate surface, surface area occupancy) and (2) digital PCR, as described elsewhere herein. The technology may be optimized to create a clinically impactful 2-target Alzheimer's Disease diagnostic assay.

### Example 4. Application of Assay Targeting p-Tau 217 and Aβ42

The compositions and methods disclosed herein may be used in an example diagnostic assay targeting p-Tau 217 and Aβ42 biomarkers as analytes. For example, the p-Tau 217 and Aβ42 may be in plasma samples or in serum samples. In an example, a lower limit of detection for p-Tau may be at least 0.5 fg/mL, a lower limit of detection for Aβ42 may be at least 50 fg/mL. Clinical samples may be used to determine the algorithm/cutoff of the assay. Recovery rate, linearity, sensitivity, specificity, or precision of the assay can be evaluated. In one example, the p-Tau 217 reference material concentration can be at least 0.01 fg/mL, and the p-Tau 217 reference material concentration can be at most 100 μg/mL. In addition, the p-Tau 217 reference material concentration can be at least 0.01 fg/mL. The Aβ42 reference material concentration can be at least 1 fg/mL, and the Aβ42 reference material concentration may be at most 100 μg/mL. In an example, the recovery rate may be calculated by comparing expected reference material concentrations spiked into serum/plasma samples to concentrations measured by the assay. A targeting recovery rate may be about 80-120%. A target for linearity may be a slope greater than 0.95 and an R² of at least 0.95. In some examples, the LLOD for p-Tau 217 may be about 50 fg/mL, the LLOD for Aβ42 may be about 50 fg/mL, the LLOD may be 2.5 standard deviations from the mean of the background signal. In some examples, the LLOQ may be the lowest concentration value were both precision (e.g., % CV≤20%) and recovery (within ±20%) criteria are met. In some examples, the serum may be spiked with a high or low amount of control material to evaluate the assay variability, the plasma may be spiked with a high or low amount of control material to evaluate the assay variability. In some examples, a high amount of control material may be about 25 μg/mL for p-Tau 217. In some examples, a high amount of control material may be about 25 μg/mL for Aβ42. In some examples, a low amount of control material may be about 5 fg/mL for p-Tau 217. In some examples, a low amount of control material may be about 500 fg/mL for Aβ42. In some examples, a target for precision may be CV of about <5%.

### Example 5. Application of Assay to Evaluate Disease State Based on Blood-Based Biomarker Measurements

The compositions and methods disclosed herein may be used in an example to evaluate disease state based on blood-based biomarker measurements. In some examples, the sensitivity can be greater than about 90% and the specificity is greater than about 90%. In some examples, the sensitivity and the specificity can detect targets that are greater than about 0.001 fg/mL in the blood. In one example, the disclosed technology may be used to run a training sample set to assess association of the biomarker measurements with disease states. The disclosed technology may be used to develop a model to call disease state based on the observed association or to evaluate the performance of the model using an additional set of samples. The sample can be about 100 samples. The sample can be from patients aged 55 and older who have mild cognitive impairment (MCI) with half of the patients with confirmed Alzheimer's Disease diagnosis.

In some examples, parameters are used to associate with the disease state, such as, an age of the patient may be a parameter taken into consideration, a gender of the patient may be a parameter taken into consideration, a BMI of the patient may be a parameter taken into consideration. In some examples, the parameters may be used to associate the assay measurements to Alzheimer's Disease diagnosis. In some examples, the parameters may be used to perform multiparametric testing and modeling based on the assay results. In some examples, the model may be used to confirm the presence of amyloid pathology or rule out amyloid pathology with high accuracy. In some examples, the intermediate group may be less than about 15-20% of the tested patients.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It can be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for analyte detection, comprising:
(a) providing a complex coupled to a surface of a substrate, wherein said complex comprises:
(i) an analyte; (ii) a first probe coupled to said analyte; (iii) a second probe coupled to said analyte, wherein said first probe or said second probe is coupled to said surface of said substrate;
(b) using said first probe and said second probe to generate a reaction product; and
(c) detecting said analyte using said reaction product,
wherein, prior to (b), said complex coupled to said surface of said substrate is washed at most once.

2. The method of claim 1, further comprising, in (c), detecting said analyte using said reaction product with a detection limit of about 0.1 fg/mL or less for detecting said analyte.

3. The method of claim 1, wherein (a)-(c) are performed in no more than about 90 minutes.

4. The method of claim 1, wherein, in (a), a density of said first probe or said second probe coupled to said surface of said substrate corresponds to no more than about 5% surface area occupancy of said surface.

5. The method of claim 1, wherein said first probe or said second probe is coupled to said surface of said substrate via a binding pair.

6. The method of claim 1, wherein said first probe or said second probe comprises an antibody, an aptamer, or an antigen.

7. The method of claim 1, wherein said analyte is a protein.

8. The method of claim 1, wherein, in (b), said reaction product comprises a fluorescence resonance energy transfer (FRET) donor or acceptor.

9. The method of claim 1, further comprising detecting said analyte using said reaction product with a specificity of greater than about 75%.

10. The method of claim 1, wherein:
(i) said first probe comprises a first binding moiety coupled to a first biomolecule;
(ii) said second probe comprises a second binding moiety coupled to a second biomolecule;
(iii) (b) comprises reacting said first biomolecule with said second biomolecule to couple said first biomolecule to said second biomolecule;
(iv) said first biomolecule is a first nucleic acid molecule and said second biomolecule is a second nucleic acid molecule;
(v) (b) comprises ligating a first element of said first nucleic acid molecule to a second element of said second nucleic acid molecule thereby generating a ligated nucleic acid molecule; and
(vi) said ligated nucleic acid molecule couples said first probe to said second probe.

11. The method of claim 10, further comprising generating a complementary nucleic acid molecule of said ligated nucleic acid molecule.

12. The method of claim 11, further comprising amplifying said complementary nucleic acid molecule of said ligated nucleic acid molecule, thereby generating said reaction product.

13. The method of claim 12, wherein said amplifying comprises performing a quantitative polymerase chain reaction.

14. The method of claim 1, wherein (c) comprises sequencing said reaction product.

15. The method of claim 1, wherein said substrate is fluorescently labeled.

16. A method for analyte detection, comprising:
(a) generating a complex, wherein said complex comprises:
(i) an analyte; (ii) a first probe coupled to said analyte; and (iii) a second probe coupled to said analyte;
(b) without washing said complex after (a), coupling said complex generated in (a) to a surface of a substrate;
(c) using said first probe and said second probe of said complex coupled to said surface of said substrate to generate a reaction product; and
(d) detecting said analyte using said reaction product.

17. The method of claim 16, further comprising, in (d), detecting said analyte using said reaction product with a detection limit of about 0.1 fg/mL or less for detecting said analyte.

18. The method of claim 16, wherein (a)-(d) are performed in no more than about 90 minutes.

19. The method of claim 16, further comprising, prior to (c), washing said complex coupled to said surface of said substrate at most once.

20. The method of claim 16, wherein: (i) said first probe or said second probe is coupled to said surface of said substrate; and (ii) a density of said first probe or said second probe coupled to said surface of said substrate corresponds to no more than about 5% surface area occupancy of said surface.

21. The method of claim 16, wherein: (i) said first probe or said second probe is coupled to said surface of said substrate; and (ii) said first probe or said second probe is coupled to said surface of said substrate via a binding pair.

22. The method of claim 16, wherein said first probe or said second probe comprises an antibody, an aptamer, or an antigen.

23. The method of claim 16, wherein said analyte is a protein.

24. The method of claim 16, wherein, in (c), said reaction product comprises a fluorescence resonance energy transfer (FRET) donor or acceptor.

25. The method of claim 16, further comprising detecting said analyte using said reaction product with a specificity of greater than about 75%.

26. The method of claim 16, wherein:
(i) said first probe comprises a first binding moiety coupled to a first biomolecule;
(ii) said second probe comprises a second binding moiety coupled to a second biomolecule;
(iii) (c) comprises reacting said first biomolecule with said second biomolecule to couple said first biomolecule to said second biomolecule;
(iv) said first biomolecule is a first nucleic acid molecule and said second biomolecule is a second nucleic acid molecule;
(v) (c) comprises ligating a first element of said first nucleic acid molecule to a second element of said second nucleic acid molecule thereby generating a ligated nucleic acid molecule; and
(vi) wherein said ligated nucleic acid molecule couples said first probe to said second probe.

27. The method of claim 26, further comprising generating a complementary nucleic acid molecule of said ligated nucleic acid molecule.

28. The method of claim 27, further comprising amplifying said complementary nucleic acid molecule of said ligated nucleic acid molecule, thereby generating said reaction product.

29. The method of claim 28, wherein said amplifying comprises performing a quantitative polymerase chain reaction.

30. The method of claim 16, wherein (d) comprises sequencing said reaction product.
